(19) United States
(12) Patent Application Publication
(10) Pub. No.: US 2016/0287063 A1
(43) Pub. Date: Oct. 6, 2016

(54) COLPOSCOPES HAVING LIGHT EMITTERS AND IMAGE CAPTURE DEVICES AND ASSOCIATED METHODS

(71) Applicant: DUKE UNIVERSITY, Durham, NC (US)

(72) Inventors: Nirmala RAMANUJAM, Durham, NC (US); Fangyao HU, Durham, NC (US); Christopher LAM, Durham, NC (US)

(21) Appl. No.: 15/038,221

(22) PCT Filed: Nov. 24, 2014
(86) PCT No.: PCT/US2014/067038

§ 371 Date: May 20, 2016

Related U.S. Application Data
(60) Provisional application No. 61/907,442, filed on Nov. 22, 2013.
(60) Provisional application No. 61/907,474, filed on Nov. 22, 2013.

(51) Int. Cl. A61B 1/06; A61B 1/012; A61B 1/303; A61B 1/00; A61B 1/05

(52) U.S. Cl. CPC A61B 1/0676; A61B 1/00082; A61B 1/05; A61B 1/0684; A61B 1/0638; A61B 1/0646; A61B 1/303; A61B 1/00045; A61B 1/00087; A61B 1/012; A61B 1/00091

(57) ABSTRACT

Colposcopes having light emitters and image capture devices and associated methods are disclosed. According to an aspect, a colposcope includes an elongate body having a distal end, a proximate end, and an axis extending between the distal end and the proximate end. The colposcope also includes a balloon attached to the elongate body and configured to be inflated to expand in a direction away from the axis of the elongate body. Further the colposcope includes an image capture device attached to the distal end of the elongate body and positioned to capture images of an area outside the elongate body. The colposcope also includes one or more light emitters attached to the distal end of the elongate body and positioned to generate and direct light towards the area outside of the elongate body.

### CROSS REFERENCE TO RELATED APPLICATIONS

This application is a 35 USC 371 application of International PCT Patent Application No. PCT/US2014/067038, filed on Nov. 24, 2014 and titled COLPOSCOPES HAVING LIGHT EMITTERS AND IMAGE CAPTURE DEVICES AND ASSOCIATED METHODS, which claims the benefit of and priority to U.S. Provisional Patent Application No. 61/907,474, filed Nov. 22, 2013 and titled SYSTEMS AND METHODS FOR DETERMINING OXYGEN SATURATION AND VASCULARITY, and U.S. Provisional Patent Application No. 61/907,442, filed Nov. 22, 2013 and titled TRANS-VAGINAL DIGITAL COLPOSCOPE AND METHODS OF USE; the disclosures of which are incorporated herein by reference in their entireties.

### GOVERNMENT RIGHTS NOTICE

This invention was made with government support under grant numbers 1R21CA162747-01 entitled “Smart Optical Sensor for Detection of Cervical Cancer in the Developing World” and 5R01EB011574-03 entitled “A Novel Optical Spectral Imaging System for Rapid Imaging of Breast Cancer,” both awarded by the National Institute of Health (NIH). Accordingly, the government may have certain rights in the invention.

### TECHNICAL FIELD

The presently disclosed subject matter relates to medical devices. Particularly, the presently disclosed subject matter relates to colposcopes having light emitters and image capture devices and associated methods.

### BACKGROUND

Numerous studies have shown that the early detection and treatment of oral and cervical cancers significantly improve survival rates. Detection of precancerous and cancerous oral lesions is mostly accomplished through visual inspection followed by the biopsy of suspicious tissue sites. For cervical cancer screening, the Papanicolau test or Pap smear is the standard of care. If the Pap smear is positive, colposcopy (visualization of the acetic acid stained cervix with a low power microscope) and biopsy are performed. An effective cancer screening and diagnostic program often requires both sophisticated and expensive medical facilities with well-trained and experienced medical staff. In developing countries, however, there is often an absence of appropriate medical infrastructure and resources to support the organized screening and diagnostic programs that are available elsewhere. Therefore, there is a critical global need for a portable, easy-to-use, reliable and low cost device that can rapidly screen for oral and cervical cancer in low-resource settings. Accordingly, there is a need for effective and low-cost equipment and techniques for cancer screening and diagnosis.

### BRIEF SUMMARY

Disclosed herein are colposcopes having light emitters and image capture devices and associated methods. According to an aspect, a colposcope includes an elongate body having a distal end, a proximate end, and an axis extending between the distal end and the proximate end. The colposcope also includes a balloon attached to the elongate body and configured to be inflated to expand in a direction away from the axis of the elongate body. Further the colposcope includes an image capture device attached to the distal end of the elongate body and positioned to capture images of an area outside the elongate body. The colposcope also includes one or more light emitters attached to the distal end of the elongate body and positioned to generate and direct light towards the area outside of the elongate body.

### BRIEF DESCRIPTION OF THE SEVERAL VIEWS OF THE DRAWINGS

The foregoing aspects and other features of the present subject matter are explained in the following description, taken in connection with the accompanying drawings, wherein:

FIG. 1 is a perspective view of a colposcope and an electronic device in accordance with embodiments of the present disclosure;

FIG. 2 is a perspective view of the colposcope and the electronic device shown in FIG. 2 with the balloon being in a deflated state;

FIGS. 3 and 4 are plan front views of a colposcope in accordance with embodiments of the present disclosure;

FIG. 5 is an exploded view of the colposcope shown in FIGS. 1 and 2;

FIGS. 6 and 7 show a colposcope in a closed position and an open position, respectively, in accordance with embodiments of the present disclosure;

FIGS. 8 and 9 show another colposcope in an open position and a closed position, respectively, in accordance with embodiments of the present disclosure;

FIGS. 10 and 11 show another colposcope in an open position and a closed position, respectively, in accordance with embodiments of the present disclosure;

FIGS. 12 and 13 are side views of an example colposcope having a cavity expander with movable members in accordance with embodiments of the present disclosure;

FIGS. 14-16 illustrate different views of an example colposcope having a cavity expander with movable members in accordance with embodiments of the present disclosure;

FIG. 17 is a perspective view of an example system including a colposcope and a control mechanism for a biopsy forcep in accordance with embodiments of the present disclosure;

FIG. 18 is a side, cross-sectional view of an example colposcope in accordance with embodiments of the present disclosure;

FIG. 19 is a side, cross-sectional view of the colposcope shown in FIG. 18 after the applicator portions have been withdrawn into respective spaces;

FIG. 20 is a side, cross-sectional diagram of another example colposcope in accordance with embodiments of the present disclosure;

FIG. 21 is a diagram of an example image capture sequence in accordance with embodiments of the present disclosure;

FIG. 22 is a block diagram of a colposcope circuit in accordance with embodiments of the present disclosure;

FIG. 23 is an exploded, side view of another example, colposcope in accordance with embodiments of the present disclosure;

FIG. 24 is a flow chart of an example ratiometric analysis for [THb] and SO₂ estimation in accordance with embodiments of the present disclosure;

FIGS. 25A-25D provide diagrams of development of analytical equations used to compute [THb] and SO₂;

FIG. 26 depicts illumination and collection parameters of the instruments used in experimental phantoms and clinical studies;

FIGS. 27A-27H show results for the simulated phantoms and the experimental phantoms;

FIGS. 28A-28H show the absolute errors of the extracted [THb] and SO₂ for the best [THb] and SO₂ ratios when using the scatter power model;

FIGS. 29A-29C show the comparison of the computational time, the mean error in [THb] extraction, and the mean error in SO₂ extraction using the scalable full spectral MC analysis and the ratiometric analysis;

FIGS. 30A and 30B show results for an in vivo cervix study;

FIGS. 31A and 31B show boxplots for SO₂ values extracted with full spectral MC analysis and the ratiometric analysis, across all measured tumor and normal sites in head and neck squamous cell carcinoma patients;

FIGS. 32A-32D show boxplots for the inverse full spectral MC or the ratiometrically extracted SO₂ of malignant and normal breast tissues;

FIG. 33A shows a full spectral MC and the ratiometrically extracted SO₂, log([THb]) that were used for building the MC and the ratiometric logistic regression models respectively;

FIG. 33B shows SO₂, log([THb]), log(μₛ′) used to build the logistic regression model for the full spectral MC analysis and the ratiometric ROC curve was built based on the SO₂ log([THb]);

FIGS. 34A and 34B show the scatter plot for the average MC extracted [THb] for the 9 tissue groups in Table 5 versus the correlation coefficients between the full spectral MC extracted and ratiometrically-extracted [THb] and SO₂; and

FIG. 35 is a flow diagram for an example colposcopy method in accordance with embodiments of the present disclosure.

### DETAILED DESCRIPTION

For the purposes of promoting an understanding of the principles of the present disclosure, reference will now be made to various embodiments and specific language will be used to describe the same. It will nevertheless be understood that no limitation of the scope of the disclosure is thereby intended, such alteration and further modifications of the disclosure as illustrated herein, being contemplated as would normally occur to one skilled in the art to which the disclosure relates.

Unless otherwise defined, all technical terms used herein have the same meaning as commonly understood by one of ordinary skill in the art to which this disclosure belongs.

In accordance with embodiments, the present subject matter relates to colposcopy. For example, colposcopes are described herein that utilize the principle of a mechanical delivery method for insertion and stabilization into the vagina for imaging of the external cervix. The imaging may produce digital, color, high-resolution images at both full field and at high magnification of areas of interest. Colposcopes described herein may include an image capture device for capture and storage of high-resolution, multimodal images of the external cervix for post-hoc analysis by medical personnel at a centralized location.

In accordance with embodiments of the present disclosure, a colposcope and electronic device may be a part of a kit provided for use by medical personnel to allow for screening of patients. Captured images may be suitably stored and processed. In an example, the images may be communicated or downloaded to a server for remote expert diagnosis. The colposcope may be suitably sterilized and subsequently re-used.

In accordance with embodiments of the present disclosure, the colposcope and an electronic operative therewith may implement a multimodal imaging technique to leverage intrinsic contrast from changes in collagen content through auto-fluorescent imaging and narrow band imaging of the neo-vascularization associated with progressively worsening cervical lesions derived from spectroscopic and ratiometric methods.

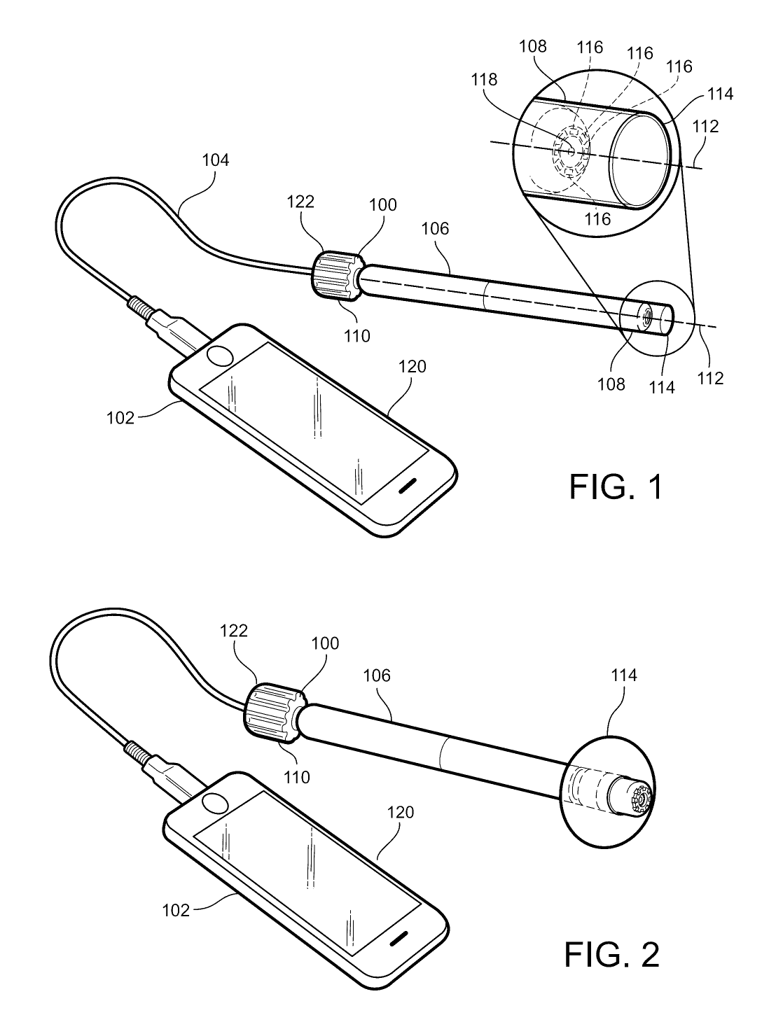

FIG. 1 illustrates a perspective view of a colposcope 100 and an electronic device 102 in accordance with embodiments of the present disclosure. The figure shows, in the circle, a detail of a distal end of the colposcope 100. Referring to FIG. 1, the colposcope 100 and electronic device 102 are communicatively connected by a cable 104. In this example, the colposcope 100 and electronic device 102 communicate in accordance with the universal serial bus (USB) standard. Alternatively, the colposcope 100 and electronic device 102 may communicate by another suitable communications standard.

The colposcope 100 includes an elongate body 106 having a distal end 108, a proximate end 110, and an axis indicated by broken line 112. The body 106 is generally tubular and rounded in shape. Alternatively, the body 106 may be of any suitable shape and size.

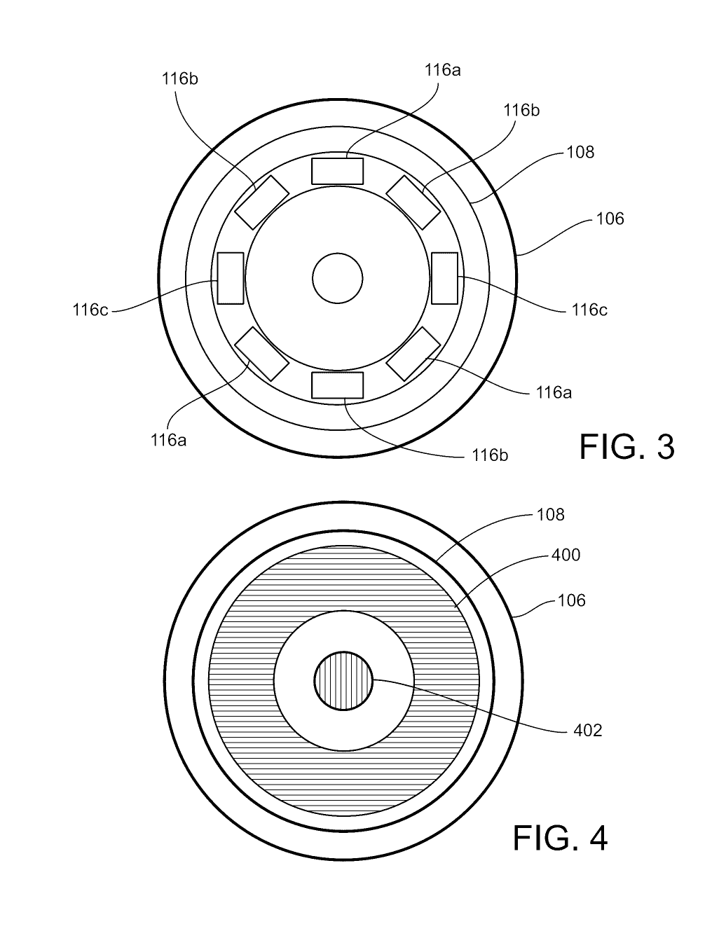

The colposcope 100 includes a balloon 114 attached to the elongate body 106. The balloon 114 is configured to be inflated to expand in a direction away from the axis 112 of the body 106. More particularly, the balloon 114 may be operative with suitable mechanisms and controls for selective inflation and deflation as described in further detail herein. The balloon 114 is shown in a deflated state in the example of FIG. 1. In contrast, FIG. 2 illustrates a perspective view of the colposcope 100 and the electronic device 102 with the balloon 114 being in a deflated state.

The balloon 114 may have one or more openings connected to a tube (not shown) for passage of air for inflation or deflation. The tube may be positioned within an interior space defined by the elongate body 106 and extend out from the proximate end 110 for connection to a mechanism to controllably inflate and deflate the balloon 114. The balloon 114 may be made of silicone rubber or a double-lumen, thin-walled membrane. Further, the colposcope 100 may include a one-way valve for retention of the dilation.

At the distal end 108, the body 106 may have attached thereto multiple light emitters 116 and an image capture device 118. In this example, the light emitters 116 are light emitting diodes (LEDs), although it should be understood that the light emitters 116 may be any suitable type of light emitter. The image capture device 118 may be a digital camera (e.g., a color CMOS sensor) configured to capture images and/or video. The image capture device 118 may be configured with one or more lenses and/or one or more filters. The light emitters 116 and the image capture device 118 may operate together for surveillance of the external cervix when the colposcope is positioned in the vaginal cavity. During image capture, the balloon 114 may be suitably inflated to expand the cavity. The balloon 114 may be automatically expanded during image capture and deflated otherwise. In an example, the balloon 114 may be suitably inflated or deflated by use of a syringe or valve. Such mechanisms may activate with sidewall compression to allow for removal of the colposcope.

The electronic device 102 may be configured to control the operation of the colposcope 100, to process captured images, and to interface with a user, such as medical personnel. In this example, the electronic device 102 is a smartphone, although it should be understood that the electronic device 102 may alternatively be any other type of computing device. It is noted that the term “electronic device” should be broadly construed. It can include any type of device capable of presenting electronic text to a user. For example, the electronic device may be a mobile device such as, for example, but not limited to, a smart phone, a cell phone, a pager, a personal digital assistant (PDA, e.g., with GPRS NIC), a mobile computer with a smart phone client, or the like. An electronic device can also include any type of conventional computer, for example, a desktop computer or a laptop computer. A typical mobile device is a wireless data access-enabled device (e.g., an iPHONE® smart phone, a BLACKBERRY® smart phone, a NEXUS ONE™ smart phone, an iPAD® device, or the like) that is capable of sending and receiving data in a wireless manner using protocols like the Internet Protocol, or IP, and the wireless application protocol, or WAP. This allows users to access information via wireless devices, such as smart phones, mobile phones, pagers, two-way radios, communicators, and the like. Wireless data access is supported by many wireless networks, including, but not limited to, CDPD, CDMA, GSM, PDC, PHS, TDMA, FLEX, ReFLEX, iDEN, TETRA, DECT, DataTAC, Mobitex, EDGE and other 2G, 3G, 4G and LTE technologies, and it operates with many handheld device operating systems, such as PalmOS, EPOC, Windows CE, FLEXOS, OS/9, JavaOS, iOS and Android. Typically, these devices use graphical displays and can access the Internet (or other communications network) on so-called mini- or micro-browsers, which are web browsers with small file sizes that can accommodate the reduced memory constraints of wireless networks. In a representative embodiment, the mobile device is a cellular telephone or smart phone that operates over GPRS (General Packet Radio Services), which is a data technology for GSM networks. In addition to a conventional voice communication, a given mobile device can communicate with another such device via many different types of message transfer techniques, including SMS (short message service), enhanced SMS (EMS), multi-media message (MMS), email WAP, paging, or other known or later-developed wireless data formats. Example functions described herein may be implemented on any suitable electronic device, such as a computer or smartphone.

The electronic device 102 may include a touchscreen display 120 and/or other user interface for interacting with a user and for present information and images. As referred to herein, a “user interface” (UI) is generally a system by which users interact with an electronic device. An interface can include an input for allowing users to manipulate an electronic device, and can include an output for allowing the system to present information (e.g., e-book content) and/or data, indicate the effects of the user's manipulation, etc. An example of an interface on an electronic device includes a graphical user interface (GUI) that allows users to interact with programs in more ways than typing. A GUI typically can offer display objects, and visual indicators, as opposed to text-based interfaces, typed command labels or text navigation to represent information and actions available to a user. For example, an interface can be a display window or display object, which is selectable by a user of a mobile device for interaction. The display object can be displayed on a display screen of an electronic device and can be selected by and interacted with by a user using the interface. In an example, the display of the electronic device can be a touch screen, which can display the display icon. The user can depress the area of the display screen at which the display icon is displayed for selecting the display icon. In another example, the user can use any other suitable interface of a mobile device, such as a keypad, to select the display icon or display object. For example, the user can use a track ball or arrow keys for moving a cursor to highlight and select the display object.

Operating environments in which embodiments of the present disclosure may be implemented are also well-known. The electronic device 102 may be communicatively connected to a remote server for communication of data and captured images for processing in accordance with embodiments of the present disclosure. Further, the electronic device 102 may suitably power the light emitters 116 and the image capture device 118 via the cable 104. In a representative embodiment, an electronic device, such as an e-book reader, is connectable (for example, via WAP) to a transmission functionality that varies depending on implementation. Thus, for example, where the operating environment is a wide area wireless network (e.g., a 2.5G network, a 3G network, or a 4G network), the transmission functionality comprises one or more components such as a mobile switching center (MSC) (an enhanced ISDN switch that is responsible for call handling of mobile subscribers), a visitor location register (VLR) (an intelligent database that stores on a temporary basis data required to handle calls set up or received by mobile devices registered with the VLR), a home location register (HLR) (an intelligent database responsible for management of each subscriber's records), one or more base stations (which provide radio coverage with a cell), a base station controller (BSC) (a switch that acts as a local concentrator of traffic and provides local switching to effect handover between base stations), and a packet control unit (PCU) (a device that separates data traffic coming from a mobile device). The HLR also controls certain services associated with incoming calls. Of course, embodiments in accordance with the present disclosure may be implemented in other and next-generation mobile networks and devices as well. The mobile device is the physical equipment used by the end user, typically a subscriber to the wireless network. Typically, a mobile device is a 2.5G-compliant device, 3G-compliant device, or 4G-compliant device that includes a subscriber identity module (SIM), which is a smart card that carries subscriber-specific information, mobile equipment (e.g., radio and associated signal processing devices), a user interface (or a man-machine interface (MMI)), and one or more interfaces to external devices (e.g., computers, PDAs, and the like). The electronic device may also include a memory or data store.

The colposcope 100 may include an interface 122 at the proximal end 110 for receipt of the tubing for the balloon 114 and any cabling for the light emitters 116 and the image capture device 118. The interface 122 may be suitably configured for connection to the cable 104.

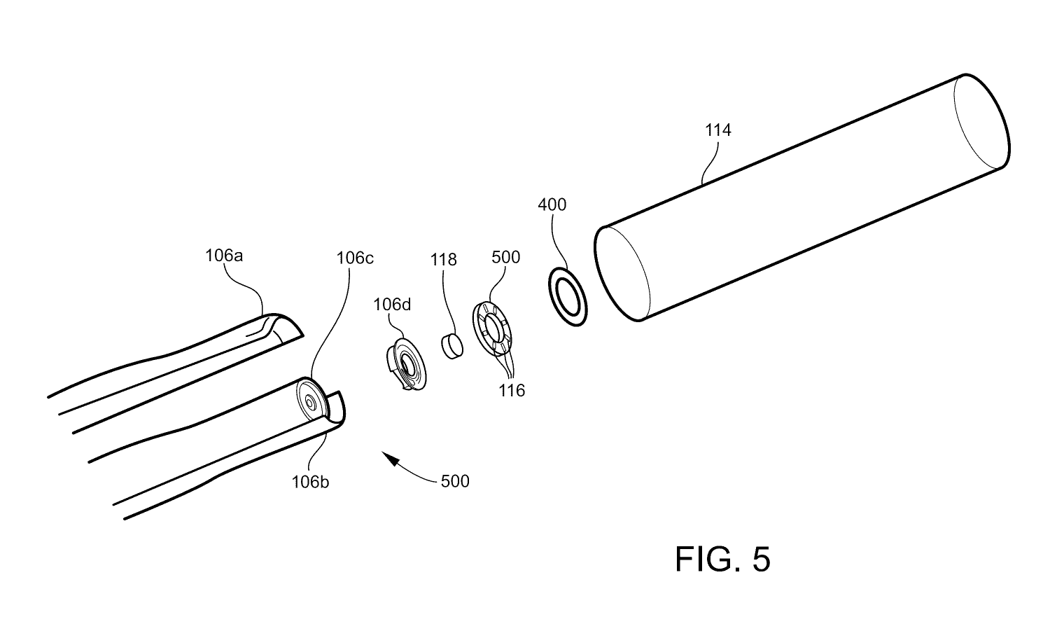

FIGS. 3 and 4 illustrate plan front views of a colposcope in accordance with embodiments of the present disclosure. Referring to FIG. 3, this example shows the distal end 108 of the colposcope body 106. Multiple LEDs 116a, 116b, 116c are attached to the distal end 108. LEDs designated 116a, 116b, and 116c are configured to generate white, blue and green light, respectively. Alternatively, the LEDs may generate any other type of light. Further, LEDs designated 116a, 116b, and 116c are configured to direct the light generally in a direction extending from the distal end 108. The LEDs 116a, 116b, and 116c are positioned to substantially surround the image capture device 118.

Now turning to FIG. 4, the figure shows placement of filters 400 and 402 with respect to the LEDs 116a, 116b, and 116c and the image capture device 118 shown in FIG. 3. Filter 400 is positioned to intercept light generated by the LEDs 116a, 116b, and 116c. Filter 402 is positioned to intercept light before receipt by the image capture device 118. The filters 400 and 402 may each be a polarizer.

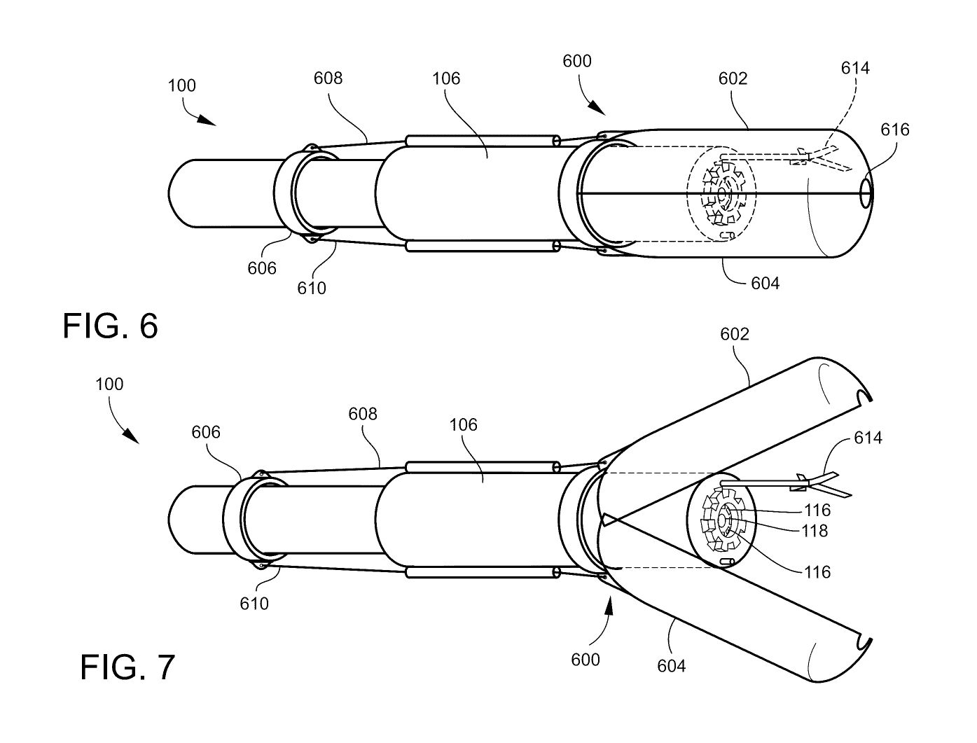

FIG. 5 illustrates an exploded view of the colposcope 100 shown in FIGS. 1 and 2. Referring to FIG. 5, the colposcope body includes outside members 106a and 106b that can fit together to form an interior space for holding an internal member 106c. Cabling and tubing may be held within an interior space formed by the internal member 106c. The outside members 106a, 106b, and 106c may form an end, generally designated 500, for receipt of and attachment to an interface 106d. The image capture device 118, a holder 502 for the light emitters 116, and the lens 400 may be held by the interface 106d. The balloon 114 may fit over and enclose an outside surface of the outside members 106a and 106b.

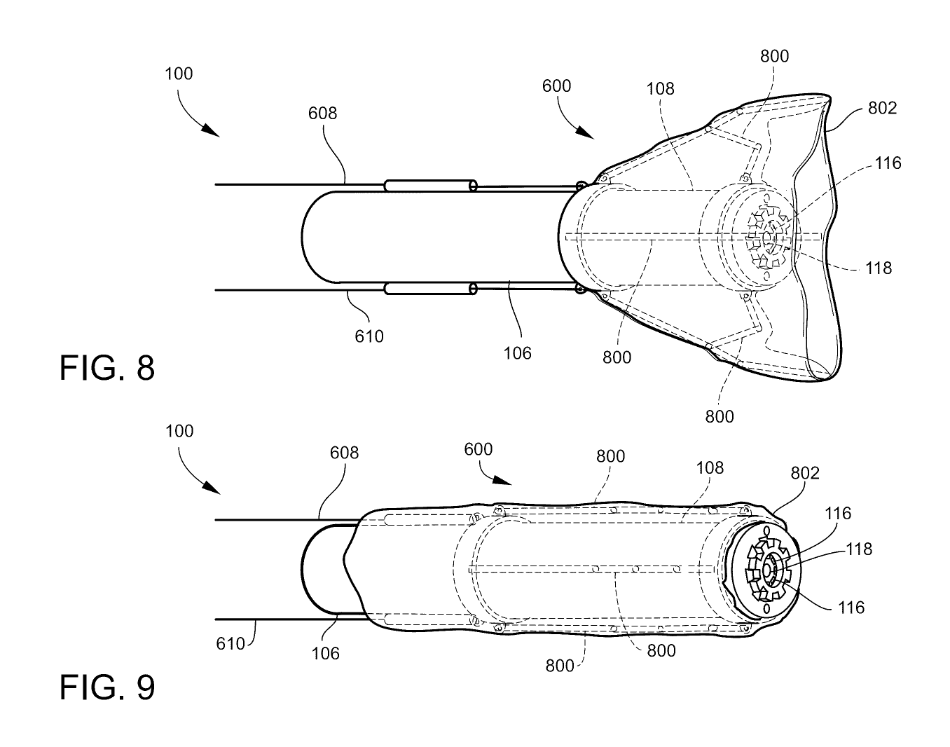

As an alternative to a balloon, a colposcope in accordance with the present disclosure may use a cavity expander having one or more members for opening to expand a human cavity. As an example, FIGS. 6 and 7 illustrate side views of an example colposcope 100 having a cavity expander, generally designated 600, with movable members 602 in accordance with embodiments of the present disclosure. Particularly, FIGS. 6 and 7 show the colposcope 100 in a closed position and an open position, respectively. Referring now to FIG. 6, cavity expander 600 is attached to the elongate body 106 and is moveable with respect thereto. The cavity expander 600 includes members 602 and 604 that are configured to be controllably positioned between a respective closed position shown in FIG. 6 and a respective open position shown in FIG. 7. When the members 602 and 604 are in the closed position, the members form a substantially tubular shape together with the body 106.

The colposcope 100 includes a mechanism for controlling movement of the members 602 and 604 between the open and closed positions. For example, the members 602 and 604 may be attached to an actuating ring 606 via wires 608 and 610 such that when the ring is moved between a position shown in FIG. 6 and a position shown in FIG. 7, the members 602 and 604 move between the opened and closed positions. The actuating ring 606 may be positioned on the body 106. When in the closed position, the members 602 and 604 may cover the light emitters 116 and the image capture device 118. When in the open position, the light emitters 116 and the image capture device 118 may be exposed to the outside for capture of images.

The colposcope 100 may include a biopsy forcep 612 attached to the distal end 108. The forcep 612 may be covered when the members 602 and 604 are in the closed position, and exposed when the members 602 and 604 are in the open position.

The members 602 and 604 may form an opening 616 at an end when in the closed position shown in FIG. 6. The opening 616 may allow the image capture device 118 to capture images through the opening 616 to allow for visual guidance.

In accordance with embodiments, the members 602 and 604 may include brushes or other features for clearing bodily fluid (e.g., mucous or blood) from the cervix. The brushes may be made of, for example, pliable, plastic fibers or the like). The brushes may be located at a tip of the members 602 and 604 such as near where the opening 616 is formed.

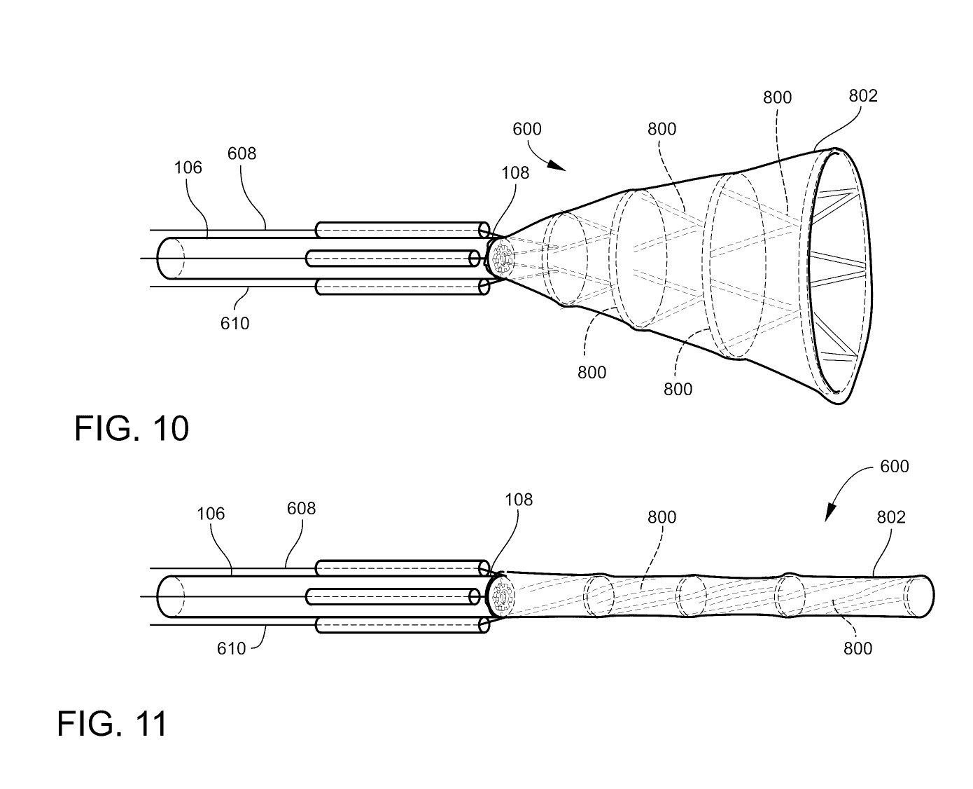

In another example, FIGS. 8 and 9 illustrate side views of an example colposcope 100 having a cavity expander, generally designated 600, with movable members in accordance with embodiments of the present disclosure. Particularly, FIGS. 8 and 9 show the colposcope 100 in an open position and a closed position, respectively. It is noted that interior components are designated by broken lines. Referring now to FIG. 8, cavity expander 600 is attached to the elongate body 106 and is moveable with respect thereto. The cavity expander 600 includes mechanical components 800 attached to a flexible membrane 802 that are configured to be controllably positioned between a respective open position shown in FIG. 8 and a respective closed position shown in FIG. 9. When the mechanical components 800 and the flexible membrane 802 are in the closed position, the members form a substantially tubular shape together with the body 106.

The colposcope 100 includes a mechanism for controlling movement of the mechanical components 800 and the flexible membrane 802 between the open and closed positions. For example, the mechanical components 800 and the flexible membrane 802 may be attached to wires 608 and 610 such movement of the wires can cause the mechanical components 800 and the flexible membrane 802 to move between the open and closed positions.

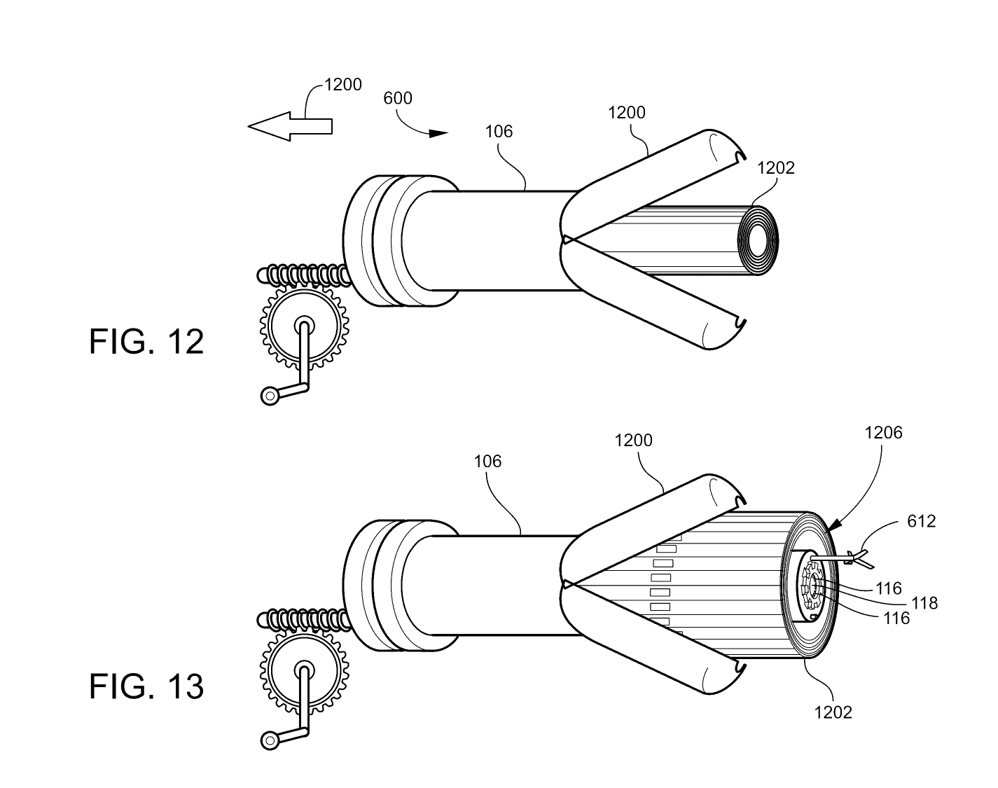

In another example, FIGS. 10 and 11 illustrate side views of an example colposcope 100 having a cavity expander, generally designated 600, with movable members in accordance with embodiments of the present disclosure. Particularly, FIGS. 10 and 11 show the colposcope 100 in an open position and a closed position, respectively. It is noted that interior components are designated by broken lines. Referring now to FIG. 10, cavity expander 600 is attached to the elongate body 106 and is moveable with respect thereto. The cavity expander 600 includes mechanical components 800 attached to a flexible membrane 802 that are configured to be controllably positioned between a respective open position shown in FIG. 8 and a respective closed position shown in FIG. 9. When the mechanical components 800 and the flexible membrane 802 are in the closed position, the members form a substantially tubular shape together with the body 106. The mechanical components 800 may be made of a rigid material such as, but not limited to, stainless steel. The flexible membrane 802 may be made of a flexible material such as, but not limited to, a double layer of PTFE material.

The colposcope 100 includes a mechanism for controlling movement of the mechanical components 800 and the flexible membrane 802 between the open and closed positions. For example, the mechanical components 800 and the flexible membrane 802 may be attached to wires 608 and 610 such movement of the wires can cause the mechanical components 800 and the flexible membrane 802 to move between the open and closed positions.

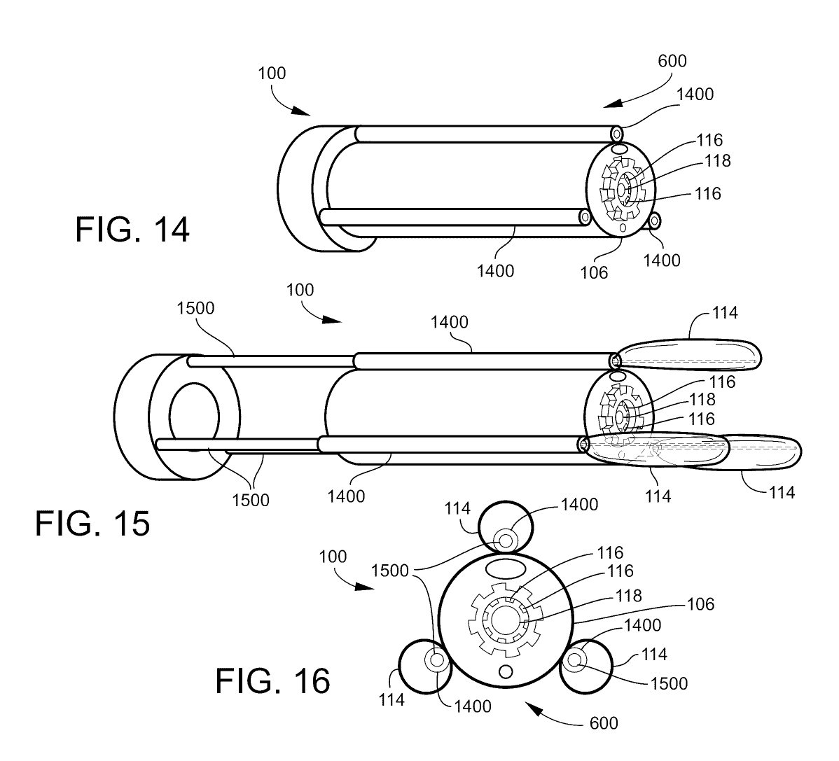

In another example, FIGS. 12 and 13 illustrate side views of an example colposcope 100 having a cavity expander, generally designated 600, with movable members in accordance with embodiments of the present disclosure. Particularly, FIGS. 12 and 13 show the colposcope 100 in a closed position and an open position, respectively. Referring now to FIG. 12, cavity expander 600 is attached to the elongate body 106 and is moveable with respect thereto. The cavity expander 600 may include a sheath 1200 configured to cover and be moved to uncover a semi-pliable, sheet of plastic 1202. The cavity expander 600 includes a mechanism 1204 configured to move the sheath 1200 between the positions shown in FIGS. 12 and 13. Referring to FIG. 12, the mechanism 1204 is being cranked or rotated to move the sheath 1200 in a direction 1206 to uncover the plastic sheet 1202. Upon being uncovered, the plastic sheet 1202 can unfurl to expand for providing an opening 1206 through which images may be captured and the forcep 612 may be exposed for use.

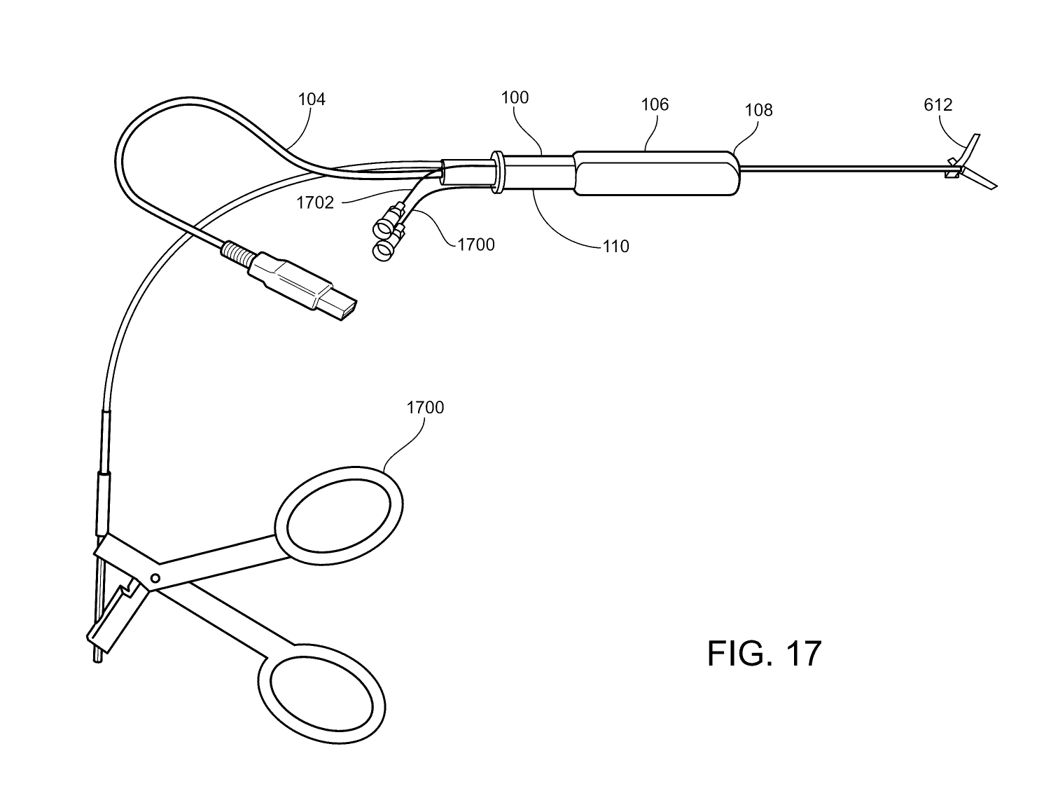

In another example, FIGS. 14-16 illustrate different views of an example colposcope 100 having a cavity expander, generally designated 600, with movable members in accordance with embodiments of the present disclosure. Particularly, FIGS. 14 and 15 illustrate side views, and FIG. 16 shows an end view. FIGS. 14-16 show the colposcope 100 at various stages for opening and closing. Referring now to FIG. 14, the sheaths 1400 may store uninflated balloons 114 and respective rods 1500 (shown in FIGS. 15 and 16) until deployment as shown in FIGS. 15 and 16.

Now turning to FIG. 15, the rods 1500 and balloons 114 may be moved by a suitable mechanism 1502 such that they move outside of the sheaths 1400. In FIG. 15, the balloons 1500 remain uninflated. FIG. 16 shows the stage in which the balloons 114 have been inflated such that the balloons 114 can expand a cavity for image capture.

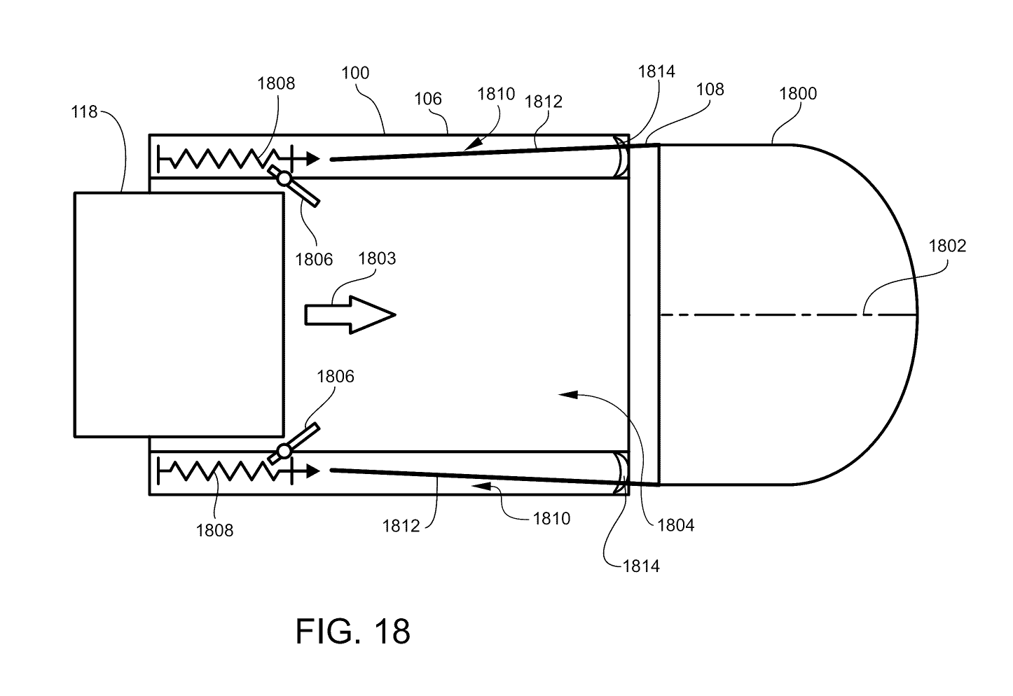

FIG. 17 illustrates a perspective view of an example system including a colposcope 100 and a control mechanism 1700 for a biopsy forcep 612 in accordance with embodiments of the present disclosure. Referring to FIG. 17, the biopsy forcep 612 may be extended, retracted, and otherwise maneuvered by operation of the control mechanism 1700. The system includes irrigation channels 1700 and 1702 for entry of and removal of fluids from a cavity area near the distal end of the colposcope 100.

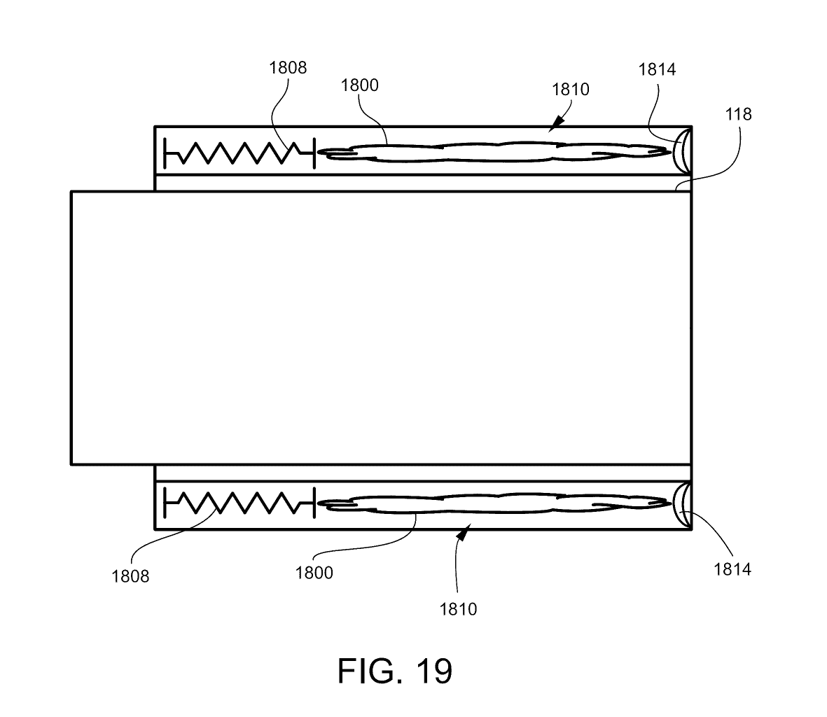

FIG. 18 illustrates a side, cross-sectional view of an example colposcope 100 in accordance with embodiments of the present disclosure. Referring to FIG. 18, the colposcope 100 of this example includes an applicator 1800 that can be used to clean excessive mucous and/or blood, and allow for retention of a human papillomavirus (HPV) sample for collection. The applicator 1800 may be a cotton pad having a perforated seam 1802. The applicator 1800 may be attached to the distal end 108 of the body 106 of the colposcope 100.

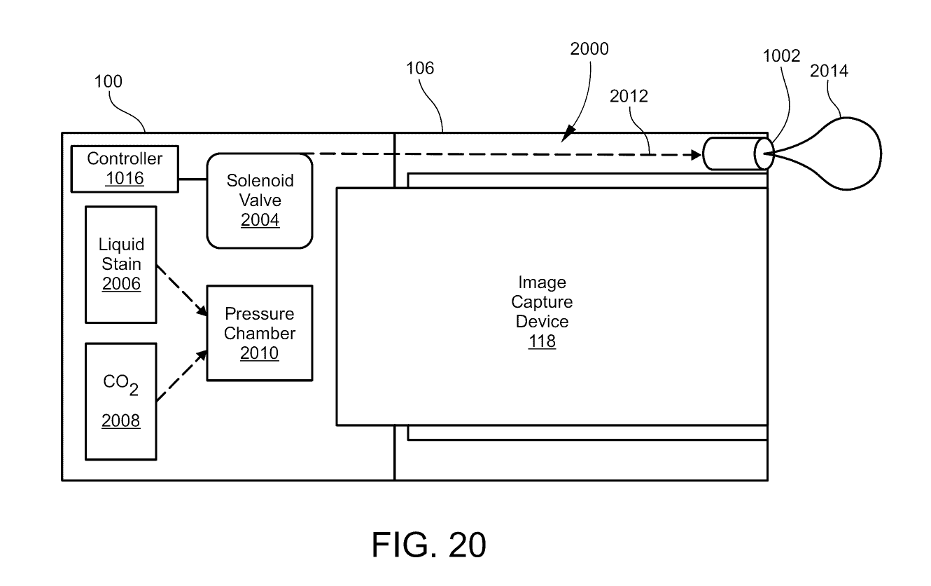

With continuing reference to FIG. 18, the applicator 1800 may be placed in the vaginal cavity and into gentle contact with the cervix for use. Subsequently, the image capture device 118 may be inserted into a moved in a direction indicated by arrow 1803 within an interior space 1804 defined by the body 106. At the image capture device 118 is moved further within the interior space 1804, an end of the image capture device 118 can engage a locking/trigger mechanism 1806 such that spring wires or shape memory components (e.g., nitinol) 1808 are activated. In turn the spring wires 1808 can retract the applicator 1800 into spaces 1810 defined within the body 106. The spring wires 1808 can be attached to respective wires 1812 that are attached to different portions of the applicator 1800. The different portions of the applicator 1800 are divided by the perforated seam 1802. Once the wires 1812 are pulled, the perforated seam 1802 may separate to result in the different portions of the applicator 1800. The spring wires 1808 may be configured such that, when activated, the different portions of the applicator 1802 are pulled into respective spaces 1810. FIG. 19 illustrates a side, cross-sectional view of the colposcope 100 shown in FIG. 18 after the applicator portions 1800 have been withdrawn into respective spaces 1810.

The colposcope 100 shown in FIG. 18 may also include diaphragms 1814 for sealing withdrawn applicator portions within the respective spaces 1810. The diaphragms 1814 may be made of PTFE or the like. The applicator portions may subsequently be processed and visually inspected.

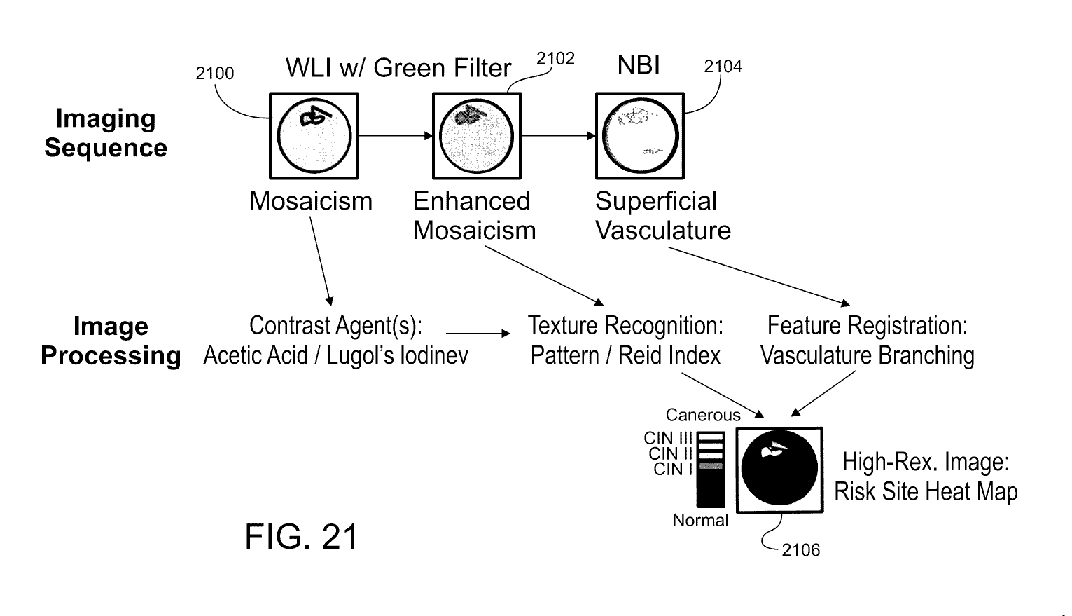

Colposcopes disclosed herein may be used for applying Lugol's iodine and/or acetic acid (5%) for aiding in the visual inspection of the cervix. In accordance with embodiments, a colposcope may include a working channel or spray channel for applying a desired amount of stain to the cervix. For example, FIG. 20 illustrates a side, cross-sectional diagram of another example colposcope 100 in accordance with embodiments of the present disclosure. Referring to FIG. 20, the body 106 of the colposcope 100 may define a channel 2000 having one end that terminates at a spray nozzle 2002 and another end that terminates at a solenoid valve 2004. In this example, liquid stain 2006 may be fed into a pressure chamber 2010, which may by pressurized by a carbon dioxide (CO₂) chamber 2008. The pressure chamber 2010 may pressurize the stain to cause the stain to move through a stainless steel tube (not shown) to the channel 2000. Once in the channel 2000, the stain is caused to move in the direction indicated by arrow 2012. The stain may then be pushed through the nozzle 2002 to generate a stain mist 2014. The stain mist 2014 may be directed by the nozzle 2002 towards the cervix for staining. The spray nozzle 2002 may aerosolize the stain droplets onto the cervix, which may be approximately 25 to 40 mm away with a target area of approximately 30 mm in diameter.

With continuing reference to FIG. 20, a controller 2016 may be operatively connected to the solenoid valve 2004 for controlling release of the stain. The controller 2016 may reside within the colposcope 100 or may be remotely located. The controller 2016 may include hardware, software, firmware, or combinations thereof configured to control stain release. For example, the controller 2016 may include one or more processors and memory.

In accordance with embodiments of the present disclosure, light emitters disclosed herein may be used for illuminating the cervix or other area of interest. For example, FIG. 1 shows a concentric ring of light emitters 116 around the image capture device 118 for providing an illuminated field for capture of images. In an example, the light emitters 116 may be LEDs, and the image capture device 118 may be CMOS sensor. The viewing angles of the light emitters 116 may be selected for overlapping fields for cross polarization to eliminate specular reflection and image saturation. A spectra of visible light may be used, because neovascularization can be a hallmark of early precancerous cervical lesions. As indicated previously, the light emitters 116 may be of different colors. In an example, LEDs may be selected and used for illumination with broad white light, a band pass of green light, and a band pass of blue light for enhanced interrogation of the underlying vasculature of the cervix. The bandwidth of the illumination may be selected based on a desired absorption spectra as will be understood by those of skill in the art. For example, narrow band LEDs may be used that are in the blue and green spectra of about 425±20 nm and 555±20 nm, respectively, which can improve imaging contrast of the cervix. LEDs of such configuration may be used as the light emitters in any of the example colposcopes described herein. Further, filters (e.g., polarizers) may be used as disclosed herein for reducing specular reflection due to the moist nature of the cervix.

Table 1 below shows a comparison of an example colposcope in accordance with embodiments of the present disclosure and a commercially-available colposcope.

TABLE 1

| | Working | Diagonal | Reso- | |
|---|---|---|---|---|
| | Distance | Field of View | lution | Illumination |
| Device | (mm) | (mm) | (lp/mm) | Type |
| Example | 30-45 | 30-45 | 14+ | Multiple |
| colposcope in | | | | wavelength |
| accordance | | | | LEDs |
| with the pres- | | | | |
| ent disclosure | | | | |
| Commercially- | 300 | 34.2 to 8 | 18+ | Halogen or |
| available | | | | White LED with |
| colposcope | | | | Bandpass Green |
| | | | | Filter |

For defogging, commercial off-the-shelf anti-fog wipes (i.e. Bausch & Lomb Fogshield XP) may be applied prior to each procedure to the outermost lens or optical window and the system has a hydrophobic optically-clear window are used to minimize obscuration of the cervix due to internal body cavity humidity induced condensation on the lens.

In accordance with embodiments of the present disclosure, a suitable imaging processing technique may be applied for the capture and analysis of cervix images. In an example, an automated imaging sequence may be implemented that transitions through the following illumination stages: white light illumination (WLI), green light illumination (green filter), and narrow band imaging (green and blue). This sequence may capture between 10 and 15 images per type of illumination strategy and may include an auto-focusing mechanism. The initial white light images may aid in characterization of the mosaicism, with enhanced mosaicism visualization with the green-light only illumination stage. Lastly, in this example, a narrow band of illumination of narrow green and blue spectra can provide important information for the vasculature of the cervix. These three components may be combined for a mathematical algorithm to aid in a probabilistic heat map for highly suspicious lesion locations as well as high-resolution color images of the cervix for reading by medical personnel, such as an obstetrician gynecologist.

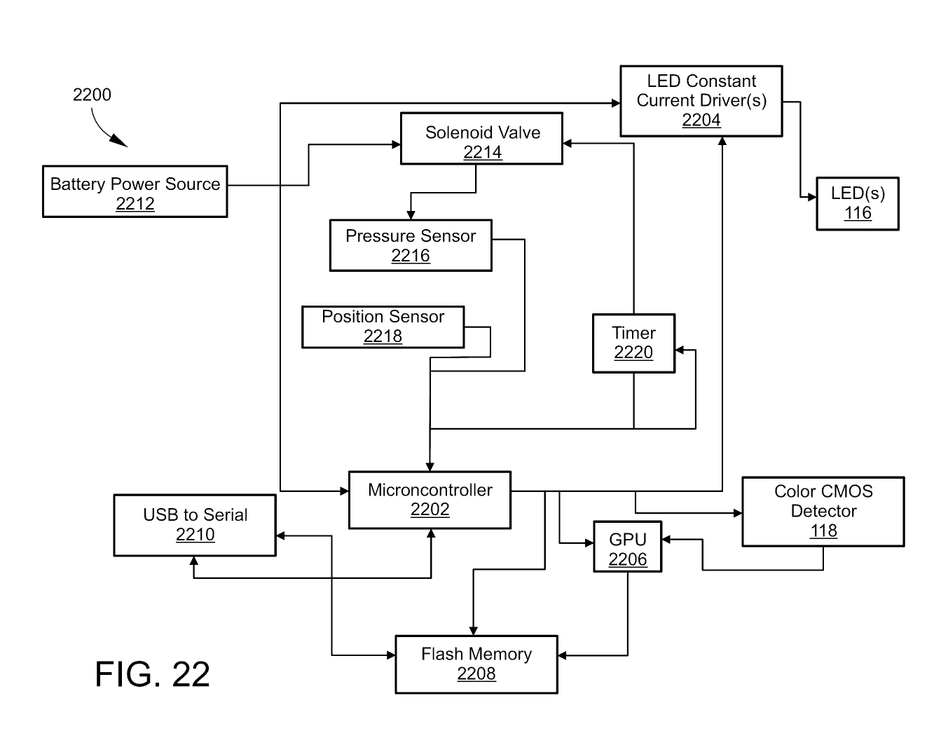

FIG. 21 illustrates a diagram of an example image capture sequence in accordance with embodiments of the present disclosure. Referring to FIG. 21, the sequence includes white light illumination 2100, followed by green spectra only 2102, and then followed by narrow band imaging 2104. These may aid in visualization of mosaicism, enhanced mosaicism, and superficial vasculature of the cervix, respectively. Mathematical image processing techniques can be utilized to enhance the digital image capture for texture recognition based on the clinical Reid index, for white light illumination and white light illumination with green filter images. This information can be combined with a feature registration-based image processing algorithm to develop a probabilistic heat map 2106 for highly suspicious regions to aid the clinician reading the results to help identify potential candidates who need further screening.

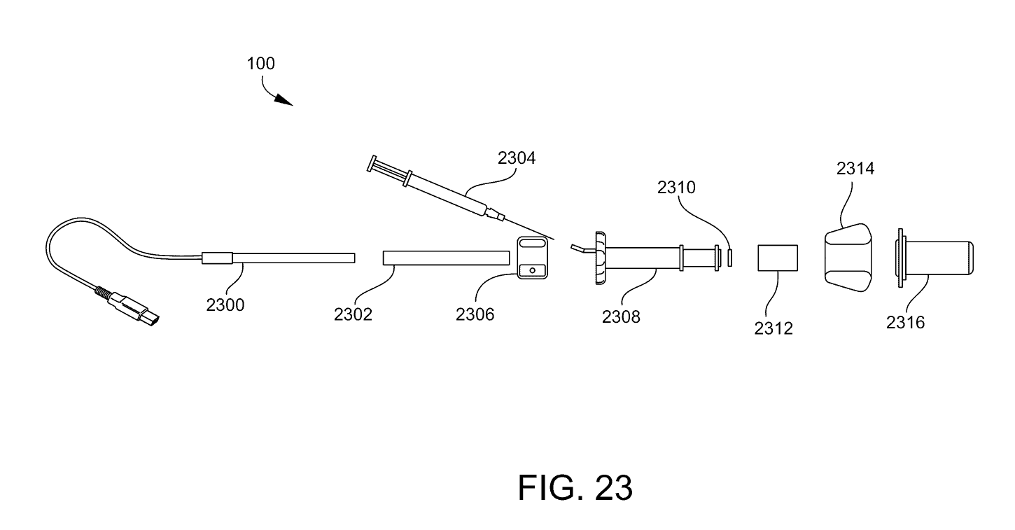

FIG. 22 illustrates a block diagram of a colposcope circuit 2200 in accordance with embodiments of the present disclosure. Referring to FIG. 22, the circuit 2200 includes a microcontroller 2202, which can provide semi-autonomous function of the colposcope after placement. The colposcope may interface with a suitable computing device implementing a suitable operating system such as, but not limited to, MICROSOFT WINDOWS®. Alternatively, the functions may be implemented by any suitable software, firmware, hardware, or combinations thereof.

With continuing reference to FIG. 22, the microcontroller 2202 may be operatively connected to one or more LED constant-current drivers 2204 for driving one or more LEDs 116 to activate or turn off in accordance with examples disclosed herein.

The microcontroller 2202 may also be communicatively connected to a color CMOS detector 118 and a graphics processing unit (GPU) 2206 (operatively connected to the detector 118) for capture of images. The microcontroller 2202 may control flash memory 2208 to store the captured images. The flash memory 2208 may store the captured images until communicated to an electronic device via a USB-to-serial interface 2210. Alternatively, the colposcope may suitably communicate captured image data via a wireless technique.

The colposcope circuit 2200 may include a battery power source 2212 configured to supply power to the LED (light source) constant current driver(s) 2204, the microcontroller 2202, and a solenoid valve 2214. Further, the colposcope circuit 2200 may include a pressure sensor 2216, a position sensor 2218, air pump (not shown) and a timer 2220. This microcontroller uses an external timer to precisely control the length of time the pump and/or solenoid valve are activate based on real-time readings from the pressure sensor to control inflation and deflation of the vaginal wall dilator mechanism and pressurize the acetic acid and/or Lugol's Iodine spray for enhancing visual contrast in the cervix.

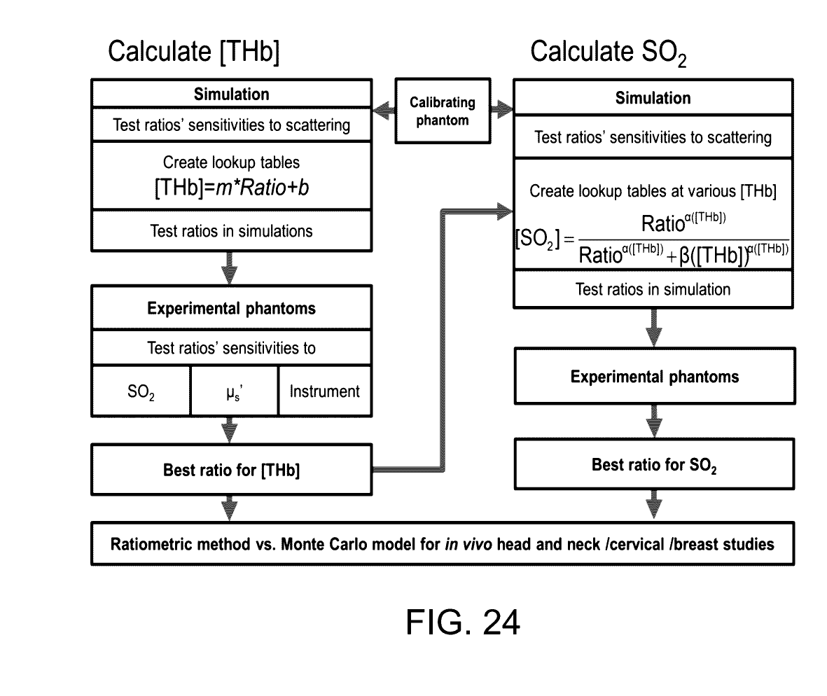

FIG. 23 illustrates an exploded, side view of another example, colposcope 100 in accordance with embodiments of the present disclosure. Referring to FIG. 23, the colposcope 100 includes a color CMOS between 2.0 to 8.0 MP detector with USB interface 2300, a stainless steel type 316L jacket 2302, and a disposable syringe, Luer lock hub, and silicon tubing to a base 2304. Further, the colposcope 100 includes a rotational adjustment component 2306 and a body 2308. The colposcope 100 includes a hydrophobic Gorilla glass anti-reflective (AR) coated window 2310 to provide sealed protective environment from biological and cleaning fluids and mitigate any potential fogging of the optical train. Further, the figure shows both the colposcope 100 also includes a deflated silicon/PET balloon 2312 and a fully inflated silicone/PET balloon 2314 used to dilate the vaginal tissue in front of the cervix in order to capture speculum free images with a full field of view of the cervix. The colposcope 100 also includes another flexible body component 2316 to gently glide the main body of the colposcope into place and is pliable to maximize patient comfort.

Disclosed herein are UV-visible (UV-VIS) diffuse reflectance spectroscopy systems, which can be used to measure tissue absorption and scattering. These systems may be used for the early diagnosis of cancers in the cervix and oral cavity. The absorption and scattering coefficients of epithelial tissues reflect the underlying physiological and morphological properties. In the UV-VIS band, the dominant absorbers in oral and cervical tissues are oxygenated and deoxygenated hemoglobin, arising from blood vessels in the stroma. Light scattering is primarily associated with cell nuclei and organelles in the epithelium, as well as collagen fibers and crosslinks in the stroma. Neoplastic tissues exhibit significant changes in their physiological and morphological characteristics that can be quantified optically. The contribution of absorption in the stromal layer can be expected to increase with neovascularization and angiogenesis, and the oxygen saturation in blood vessels is expected to decrease as the neoplastic tissue outgrows its blood supply. Stromal scattering can be expected to decrease with neoplastic progression due to degradation of extracellular collagen networks. However, epithelial scattering can be expected to increase due to increased nuclear size, increased DNA content, and hyperchromasia. UV-VIS diffuse reflectance spectroscopy has a penetration depth that can be tuned to be comparable to the thickness of the epithelial layer or deeper to probe both the epithelial and stromal layers.

In accordance with embodiments disclosed herein, a UV-VIS diffuse reflectance spectroscopy system is provide having a colposcope geometry that is most sensitive to changes in the stroma and a scalable inverse Monte Carlo (MC) reflectance model to rapidly measure and quantify tissue optical properties. In one study, it was shown that a spectroscopic system and the MC model may be used to identify optical biomarkers that vary with different grades of cervical intraepithelial neoplasia (CIN) from normal cervical tissues. In another study, total hemoglobin was found to be statistically higher in high-grade dysplasia compared with normal and low grade dysplasia (P,0.002), whereas scattering was significantly reduced in dysplasia compared with normal tissues (P,0.002). Further, in another study, the same UV-VIS diffuse reflectance spectroscopy system was applied in an in vivo in which 21 patients with mucosal squamous cell carcinoma of the head and neck were evaluated. All 21 patients underwent panendoscopy and biopsies were taken from the malignant and the contralateral normal tissues. Diffuse reflectance spectra were measured prior to biopsy. The vascular oxygen saturation (SO₂) was found to be statistically higher in malignant tissues compared to non-malignant tissues (P=0.001).

It is known that the most efficient and effective strategy for the prevention of advanced cervical or oral cancers in resource-limited settings is to see and treat the patient in a single-visit, thus obviating the need for a multi-tiered system such as that in the U.S. where screening, diagnosis, and treatment entail three or more visits to the healthcare facility. For example, guidelines have been written by the Alliance for the Prevention of Cervical Cancer (APCC) on strategies for screening cervical cancer in resource-limited settings. Their recommendation is visual inspection with acetic acid (VIA), followed by treatment of the precancerous lesions using cryotherapy (freezing), which can be carried out by physicians, nurses or midwives. An effective screening/diagnostic strategy that can allow for immediate treatment intervention needs to be able to survey the entire region of interest. Further, the detection strategy should be minimally affected by operator bias or subjective interpretation of images collected from the region of interest. Systems disclosed herein can enable quantitative determination of tissue physiological endpoints, but may be limited to evaluating localized regions of the tissue. To survey the entire field of view, it is important to scale the single-pixel fiber-based system into an imaging platform and develop algorithms that can quantify these spectral images. However, development of simple imaging systems may require a significant consolidation of the number of wavelengths, so that imaging spectrographs and broad-band thermal sources can be replaced by simple cameras and LEDs.

Systems disclosed herein can use a ratiometric analysis for the quantitation of tissue SO₂ and total hemoglobin concentration ([THb]) using a small number of wavelengths in the visible spectral range as a strategy for implementation of rapid surveillance of pre-cancers and cancers in a screening population in resource-limited settings. For example, the analysis may be used by colposcopes and associated electronic devices disclosed herein. Ratiometric analyses may be used to compute [THb] or SO₂ from reflectance spectra. For example, ratiometric analyses may be used to extract SO₂ using ratios at two wavelengths, one where the local differences between the extinction coefficients of oxy- and deoxy-hemoglobin are maximal, and one isosbestic wavelength, where the extinction coefficients of oxy- and deoxy-hemoglobin are the same. A ratiometric analysis is disclosed which computes reflectance ratios at the isosbestic wavelengths of hemoglobin, and this analysis may be used to rapidly calculate [THb] independent of tissue scattering and SO₂. For this particular ratiometric analysis, the ratio of the intensities at one visible wavelength (452, 500, or 529 nm) to one ultraviolet wavelength (390 nm) from a diffuse reflectance spectrum was used to extract [THb] using a linear analytical equation. This analysis may require an ultraviolet source, which is relatively expensive compared to ubiquitous visible wavelength light sources. Herein, an analytical ratiometric analysis is provided for extracting both [THb] and SO₂ in the visible wavelength range. It utilizes two or more intensities at different wavelengths from a diffuse reflectance spectrum and calculates appropriate ratios from them. The derived ratios may then be converted to [THb] or SO₂ using analytical equations. The analysis, in one example, utilizes only three wavelengths (539, 545 and 584 nm), all in the visible part of the spectrum where light emitting diodes (LEDs) are readily available. This ratiometric analysis was tested with full spectral MC simulations and experimental phantoms to ensure minimal sensitivity to scattering. In addition, the ratiometric analysis may also account for [THb] when computing SO₂.

In an example study, wavelengths were chosen from 500 nm to 600 nm (visible spectrum) in order to leverage relatively low priced light sources such as LEDs. In addition, deoxy- and oxy hemoglobin have distinct absorption features in the visible spectrum. Five isosbestic wavelengths and five other wavelengths where the difference of extinction coefficients between deoxy- and oxy-hemoglobin are largest were used to calculate [THb] and SO₂, respectively. Table 2 below lists these wavelengths, which provide a total of ten possible combinations (pairs of isosbestic wavelengths), at which ratios were tested for extraction of [THb] and 25 wavelength combinations at which the reflectance ratios were tested (one isosbestic and one maximal difference wavelength) for extraction of SO₂.

TABLE 2

| Isosbestic Wavelengths | Wavelengths for Oxygen |
|---|---|
| for [THb] | Saturation (SO₂) |
| (nm) | (nm) |
| 500 | 516 |
| 529 | 539 |
| 545 | 560 |
| 570 | 577 |
| 584 | 593 |

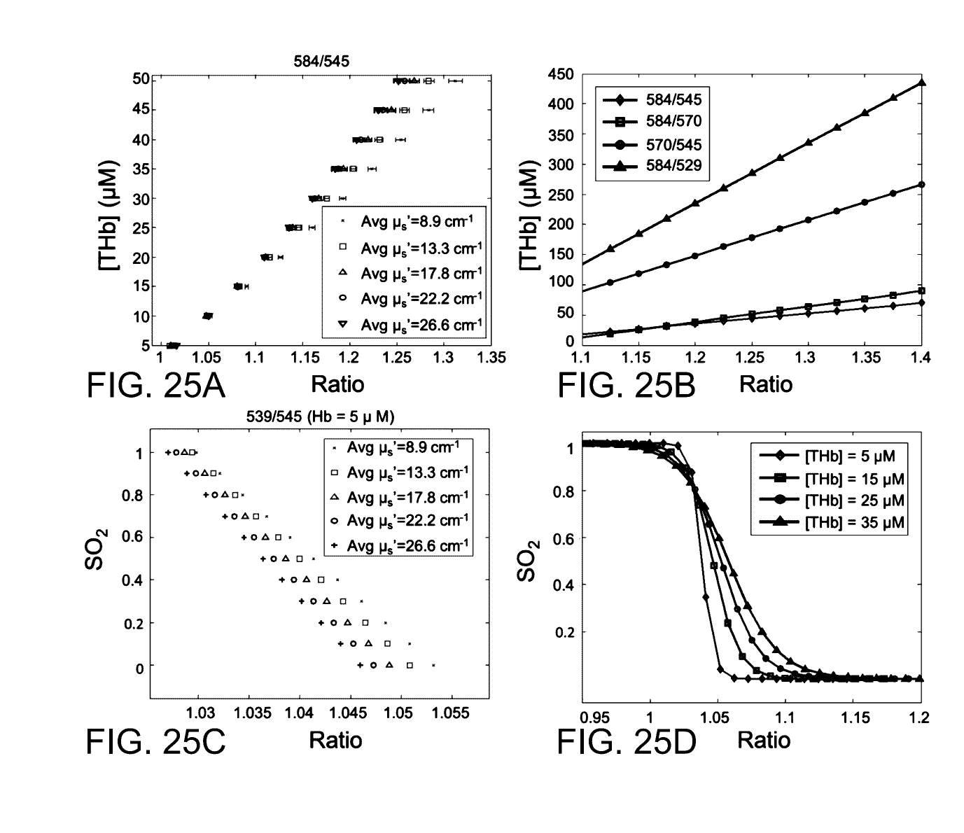

FIG. 24 illustrates a flow chart of an example ratiometric analysis for [THb] and SO₂ estimation in accordance with embodiments of the present disclosure. Referring to FIG. 24, the figure briefly provides an overview of the ratiometric analysis including the steps involved in the selection of the best ratios for [THb] and SO₂. Extractions of [THb] and SO₂ may be achieved in two steps. First, the reflectance ratio comprises isosbestic wavelengths was used to extract [THb]. This may be achieved by converting the reflectance ratio into [THb] using a linear equation. For each ratio at isosbestic wavelengths, independent sets of the coefficients m and b were generated using MC simulations. Next, the reflectance ratio at one isosbestic wavelength and one maximal-difference wavelength may be converted into an SO₂ value using a non-linear equation using the a (THb) and b (THb) coefficients. These coefficients may be generated using MC simulations for each of the 25-reflectance ratios at every simulated [THb]. The extracted [THb] from the first step may be used to select the appropriate non-linear logistic equation to convert the ratio of the isosbestic to maximum difference wavelength into the SO₂ value. After the equations for [THb] and SO₂ are developed, the ratiometric analysis may be validated with experimental tissue mimicking phantoms. To show the clinical utility of this analysis and its independence to changes in instrumentation, the extractions using the selected ratios may subsequently be compared with those using the full spectral MC analysis in three different clinical studies carried out with different optical systems.

Analytical equations to convert appropriate ratios into [THb] and SO₂ values may be determined using full spectral MC simulations. A suitable forward full spectral MC model may be used to generate 24805 unique diffuse reflectance spectra. These reflectance spectra may serve as the simulated master set. Diffuse reflectance spectra may be simulated by calculating the absorption and scattering spectrum between 350-600 nm. The absorption coefficients may be calculated with the assumption that oxy- and deoxy-hemoglobin are the dominant absorbers in tissue. The sum of these two absorber concentrations may provide the resulting [THb], which was varied between 5 and 50 mM in increments of 0.1 mM in the master set. The concentration of each hemoglobin species may be varied to span the range of SO₂ values from 0 to 1, in steps of 0.1. The reduced scattering coefficients, ms′, across the spectral range may be determined using Mie theory for 1 mm polystyrene microspheres. Five different scattering levels may be generated by increasing the number density of sphere concentrations. The wavelength-averaged (between 350,600 nm) mean reduced scattering coefficients for these five scattering levels were 8.9, 13.3, 17.8, 22.2, and 26.6 cm⁻¹. The resulting master set consisted of 24805 reflectance spectra, which represent the combination of all possible [THb] levels, with all SO₂ levels, and all scattering levels (45161165=24805). These optical properties are similar to those previously used. The simulated reflectance spectra for the master set may be created for a fixed fiber-probe geometry in a suitable manner. Finally, an experimentally measured diffuse reflectance spectrum with the same fiber-geometry may be used as a “reference” to calibrate the scale of the simulated spectra to be comparable to that of measured spectra.

To study the impact on extraction accuracy of the ratiometric analysis with increasing spectral bandpasses, additional bandpasses in the master set were simulated. The reflectance spectra were simulated for three different bandpasses (2 nm, 3.5 nm and 10 nm full width-half-maximum (FWHM) bandwidths) and resulted in 3 modified master diffuse reflectance sets (each containing 24,805 spectra). This was done by assuming each wavelength had a certain Gaussian bandpass of specified FWHM. Specifically, the reflectance at each wavelength in the simulated spectrum was convolved with a Gaussian distribution function with the specific bandpass. Equations to convert reflectance ratios into [THb] and SO² were then generated separately for each of the three bandpass-modified master diffuse reflectance spectral sets.

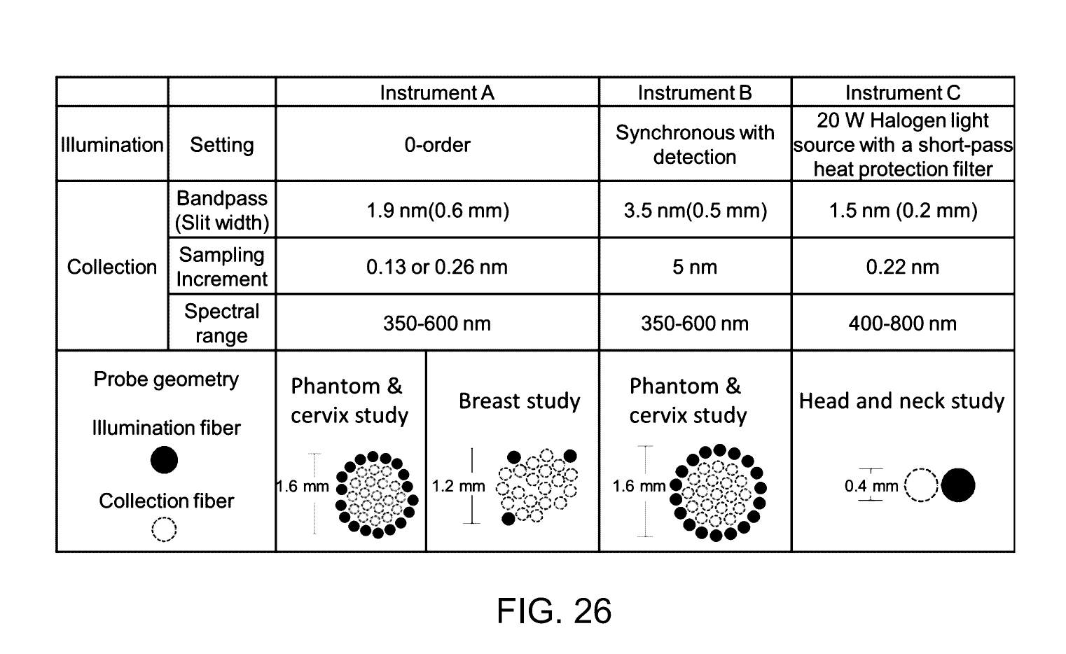

FIGS. 25A-25D describe development of analytical equations used to compute [THb] and SO₂. Particularly, the figures show steps for calculating the analytical equations. FIG. 25A shows generating reflectance with various optical properties using forward analysis and derived Hb ratios. The horizontal error bars show the standard deviation of the ratios at SO₂ levels from 0 to 1. The spreads are small because the ratios are derived from isosbestic points. FIG. 25B shows example linear analytical equations of 584/545, 584/570, 570/545, and 584/529 for [THb] estimation. FIG. 25C shows calculating SO₂ ratios with several scattering levels at one [THb]. FIG. 25D shows Hill curve equations were generated at doi:10.1371/journal.pone.0082977.g002. For [THb] extraction, the reflectance ratio at a given wavelength-pair was computed from every simulated reflectance spectrum that had a fixed [THb]. Thus, there were 55 values for a given [THb] wavelength-ratio (across the 5 scattering levels and 11 SO₂ levels). Eleven of these values were averaged across SO₂, for each scattering level. For each of the ten isosbestic wavelength-pairs, the dependence of the reflectance ratio on [THb] was plotted across all SO₂ levels and each scattering level, as shown in FIG. 25A. Although the analysis consisted of 5-50 mM [THb] in steps of 0.1 mM, only 10 of the 451 [THb] levels are shown in the figure for easier interpretation of the data points. The dependence of the reflectance ratio was evaluated for a given wavelength-pair on tissue SO₂ and scattering. The horizontal error bars at each scattering level show the spread of the reflectance ratio due to varying SO₂ levels from 0 to 1. This reflects the sensitivity of the ratio to changes in SO₂. The spread in the different symbols at each [THb] reflects the sensitivity of the ratio to scattering. The reflectance ratios at each [THb] were averaged across the 5 scattering levels and the 11 SO₂ levels, and a linear analytical equation was generated for the averaged ratios. FIG. 25B shows the linear analytical equations for 584/545, 584/570, 570/545, and 584/529 as examples.

In order to convert the reflectance ratio computed at a given SO₂ wavelength-pair into an SO₂ value, a non-linear logistic (Hill curve) equation was used. A unique Hill equation was generated for each of the 451 [THb] (5-50 mM in 0.1 increment steps) in the modified master set. The reflectance ratio for a given SO₂ wavelength-pair, at a given [THb], was averaged across the five scattering levels (FIG. 25C). This resulted in 11 averaged ratios for each SO₂ wavelength pair, at each [THb]. The Hill coefficients were generated by fitting the 11 averaged ratios to the logistic equation. Since a total of 451 different [THb] values were used in the simulations, 451 different equations were generated for each SO₂ wavelength pair. FIG. 25D shows the example figures of the Hill curves generated from the averaged ratios at different [THb] for 539/545.

A total of 8 sets of reflectance spectra were used to validate the ratiometric analysis. The optical properties and collection parameters for these 8 phantom sets are summarized in Table 3 shown below.

TABLE 3

| | | | Bandpass | | [THb] | |
|---|---|---|---|---|---|---|
| Set | Type | Instrument | (nm) | SO₂ | (μm) | <μₛ′> (cm⁻¹) |
| 1 | Simulation | — | 2 | 0-1 | 5-50 | 8.9-26.6 |
| 2 | Simulation | — | 5 | 0-1 | 5-50 | 8.9-26.6 |
| 3 | Simulation | — | 10 | 0-1 | 5-50 | 8.9-26.6 |
| 4 | Experiment | A | 2 | 0-1 | 14.8 | 12.6 |
| 5 | Experiment | A | 2 | 1 | 6.4-14.3 | 13-21.7 |
| 6 | Experiment | A | 2 | 1 | 5.9-35.2 | 17.3-23.6 |
| 7 | Experiment | B | 3.5 | 1 | 7.3-16.2 | 14.3-21.9 |
| 8 | Experiment | B | 3.5 | 1 | 5.0-50.0 | 13.0-21.9 |

Phantom sets 1-3 were simulated with the scalable MC model, as described above. Phantom sets 4-8 were experimentally measured data. Briefly, Phantom Set 4 consisted of 51 phantoms with varying SO₂ levels but with a fixed [THb] (14.8 mM), and μs″ level (12.6 cm⁻¹). Phantom Set 5 consisted of two subsets of phantoms with a low scattering level (μs′=13.5 cm⁻¹) and high scattering level (μs′=22.52 cm⁻¹). Each set in Phantom Set 5 consisted of 4 phantoms. Each phantom in the low scattering level was paired with a phantom in the high scattering level and the [THb] value of each paired phantom was the same. The standard deviation of the reflectance for each wavelength-pair in each paired phantoms were computed. Phantom Set 6 consisted of 13 phantoms with increasing [THb] from 5.86-35.15 mM. The averaged μs′ levels decreased for each phantom from 23.63 to 17.30 cm⁻¹. A second instrument was used to measure the phantoms for Phantom Set 7 and Set 8 to validate the instrument independence of the ratiometric analysis. Phantom Set 7 was similar to Phantom Set 5 in that it contained two sets of 4 phantoms with low and high scattering levels (μs′=13.5 cm⁻¹ and 22.89 cm⁻¹ respectively) and paired phantoms from each level contained the same [THb]. The standard deviation of the reflectance for each wavelength-pair in each paired phantoms were also computed. Phantom Set 8 consisted of 16 phantoms with increasing [THb] from 5-50 mM. The μs′ level of each phantom was lower than the previous phantom, ranging from 28.56 to 17.02 cm⁻¹, due to serial dilutions of the phantom solution. The combination of all of these experimental tissue phantoms measured serves to determine the best ratios to estimate [THb] and SO₂ for a wide range of optical properties measured by different instruments.

The ratiometric analysis was first tested on the simulated reflectance. Linear analytical equations for [THb] ratios and the non-linear logistic equations for SO₂ ratios were generated from Phantom Sets 1-3. The extracted values of [THb] using the ratiometric analysis were compared to the true values for each diffuse reflectance spectrum and the absolute errors between the predicted and true values were calculated. Next, the sensitivity of each [THb] ratio to scattering was computed using the standard deviation of the reflectance ratio at each [THb].

The calculation of [THb] using the ratiometric analysis was also validated in Phantom Sets 4-8. Since every reflectance spectrum simulated by the MC model needs to be scaled by a calibrating phantom, the choice of the calibrating phantom can introduce systematic errors. To account for these effects on the extracted [THb], 3 different phantoms in Phantom Set 4, Set 6 and Set 8 and 2 different phantoms in Sets 5 and 7 were selected as the calibrating phantoms. The SO₂, [THb] and μs′ of the calibrating phantoms are summarized in Table 4 below.

| | | | Avg. μₛ′ (350~600 nm) | | |
|---|---|---|---|---|---|
| | | | μₛ′ = 6 | μₛ′ = 2 | μₛ′ = 10 |
| [THb] | | Scattering | (cm⁻¹) at | (cm⁻¹) at | (cm⁻¹) at |
| (μm) | SO₂ | Power | 600 nm | 600 nm | 600 nm |
| 5-50 | 0-1 | 0.2 | 2.05 | 6.17 | 10.28 |
| 5-50 | 0-1 | 0.4 | 2.11 | 6.34 | 10.57 |
| 5-50 | 0-1 | 0.6 | 2.17 | 6.52 | 10.87 |
| 5-50 | 0-1 | 0.8 | 2.24 | 6.71 | 11.18 |
| 5-50 | 0-1 | 1 | 2.30 | 6.91 | 11.51 |
| 5-50 | 0-1 | 1.2 | 2.37 | 7.11 | 11.84 |
| 5-50 | 0-1 | 1.4 | 2.43 | 7.32 | 12.20 |
| 5-50 | 0-1 | 1.6 | 2.51 | 7.54 | 12.56 |
| 5-50 | 0-1 | 1.8 | 2.59 | 7.77 | 12.94 |
| 5-50 | 0-1 | 2 | 2.67 | 8.00 | 13.34 |

Each time a calibrating phantom was selected, a new master set of reflectance was generated with the scalable MC model, and new coefficients for analytical equations were generated from these phantom sets. The generated analytical equations were used to extract the [THb] or SO₂ values in the same experimental phantom sets from which the calibrating phantoms were selected. This ensured that the systematic errors or titration errors in one experimental phantom study were restricted to the same experimental phantom study and were not carried to another experimental phantom study. The probe geometries and bandpasses for the simulated master sets were matched to the experimental system. The ratiometrically extracted [THb] were compared to the MC extracted [THb] of the experimental phantoms for each phantom in Sets 4-8 to compute the absolute errors. The ratio spreads of the ten possible isosbestic wavelength pairs were computed from the paired phantoms in Set 5 and Set 7. The best ratio for [THb] was determined from the error and ratio spread rankings both with the simulated data and with the experimental data.

The ratiometric analysis for SO₂ was validated in Phantom Set 4, which consisted of phantoms with varying SO₂ levels. For each experimental phantom in this set, [THb] was first computed using the best isosbestic wavelength-pair using the ratiometric analysis. This extracted [THb] was then used to select the corresponding Hill curve coefficients for a given SO₂ wavelength-pair. The reflectance ratio of each SO₂ wavelength-pair was first computed and then converted to a SO₂ value with the corresponding Hill curve coefficients. The ratiometrically extracted SO₂ values were compared against the SO₂ values measured with a pO₂ electrode. To evaluate the sensitivity of each SO₂ ratio to scattering, the reflectance ratios of each SO₂ wavelength-pair were first computed in every phantom of Phantom Sets 5 and Set 7. The standard deviations were then computed from each paired reflectance ratios for each SO₂ wavelength-pair since only the scattering was different within each paired phantom. The derived standard deviations from every paired phantom in Phantom Set 5 and Set 7 were averaged for each SO₂ wavelength-pair.

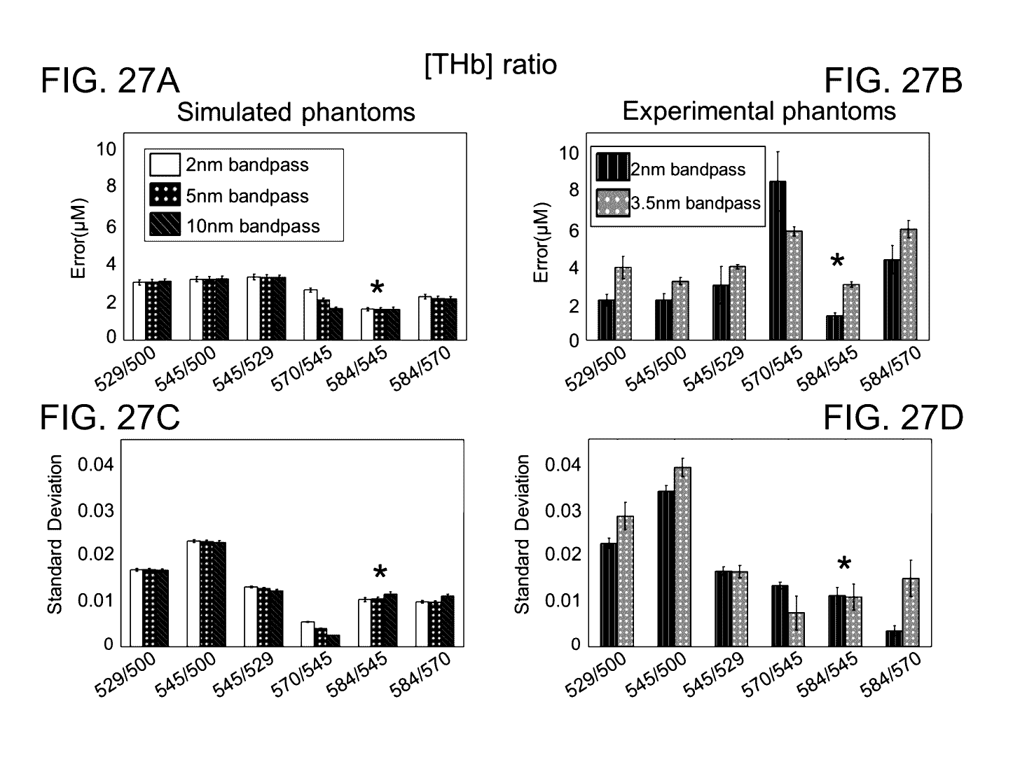

Three instruments were used to validate the ratiometric analysis in this manuscript. Instrument A was used in the experimental phantom studies (Set 4-6) and in an in vivo cervical study. Instrument B was also used in the experimental phantom studies (Set 7-8), and also in the in vivo cervical study and in an in vivo breast cancer study. Instrument C was used for an in vivo head and neck cancer study. The details of Instruments A, B and C and the probe geometries were determined. Briefly, Instrument A consisted of a 450 W xenon (Xe) arc lamp (JY Horiba, Edison N.J.), double excitation monochromators (Gemini 180, JY Horiba, Edison, N.J.), and a Peltier-cooled open electrode charge-coupled device (CCD) (Symphony, JY Horiba, Edison, N.J.). Instrument B was a fibercoupled spectrophotometer (SkinSkan, JY Horiba, Edison, N.J.), which consisted of a 150 W Xe arc lamp, a double-grating excitation monochromator, an emission monochromator, and an extended red photomultiplier tube (PMT). Instrument C was a portable system, which consisted of a 20 W halogen lamp (HL2000HP; Ocean Optics, Dunedin, Fla.), heat filter (KG3, Schott, Duryea, Pa.), and an USB spectrometer (USB4000, Ocean Optics, Dunedin, Fla.). Illumination and collection for all instruments were achieved by coupling to fiber optic probes. The instrument parameters are listed in FIG. 26, which depicts illumination and collection parameters of the instruments used in experimental phantoms and clinical studies.

The power law (μₛ′=a·λ^{-b}) was used to model the reduced scattering coefficients where a determines the overall magnitude of scattering, 1 is wavelength, and b is the scattering power. A new set of 1500 reflectance spectra (10 [THb] levels, 5 SO₂ levels, and 10 different scattering powers with the scattering values equal to 2, 6, or 10 cm₂⁻¹ at 600 nm) were simulated with the forward Monte Carlo model using the scattering coefficient generated from the power law. The scattering power was varied from 0.2 to 2 with steps of 0.2. The [THb] were range from 5 to 50 mM in steps of 5. The SO₂ levels were range from 0 to 1 with increment of 0.25. Table 4 summarizes the optical properties used for testing the ratiometric analysis with various scattering powers. The [THb] and the SO₂ were extracted with the ratiometric analysis for the best ratios determined herein. The absolute [THb] and SO₂ errors were computed. In addition, the scattering powers of the clinical data in this manuscript were computed by fitting the Monte Carlo-extracted wavelength-dependent scattering coefficients to the scatter power model.

To compare the computational performance of the ratiometric analysis and the full spectral MC analysis for extraction of [THb] and SO₂, 100 diffuse reflectance spectra with randomly selected [THb] and SO₂ values were simulated with the forward MC model. Random white noise was also added to each simulated reflectance spectrum before the fitting process. The amplitude of the generated random noise was limited to two percent of the difference between the simulated maximum and the minimum values of each reflectance spectrum. The noise level was determined from a previous study in which the worst SNR of instrument A is 44.58 dB. This means the amplitude of the noise is about two percent of the amplitude of the signal. These spectra were then analyzed using both the inverse full spectral MC analysis and the ratiometric analysis. The ratiometric analyses on these samples used the best ratios, which are described in the subsequent sections of this manuscript, for [THb] and SO₂. The extracted [THb] and SO₂ values for the full spectral MC analysis and the ratiometric analysis were compared to the expected (input) values and absolute errors were computed. The data processing time for both analyses were also compared.

To test the robustness of the ratiometric analysis in in vivo clinical settings, the ratiometric analysis was applied in three separate studies conducted on three different tissue sites. These clinical studies used diffuse reflectance spectroscopy to differentiate normal versus malignant or precancerous tissues in vivo in the cervix, in the breast, and in the head and neck. The samples from these studies represent different optical absorption scenarios. Head and neck and breast tissues have relatively high [THb] while the cervix has [THb] values at the lower end of the spectrum. The ranges of [THb] from previous results were 2.6-208.9 mM, 0.79-63.7 mM and 0.99-44.06 mM, for the head and neck, breast, and cervical tissues, respectively. In addition, breast tissue contains not only [THb] but also b-carotene as an additional absorber. Data previously collected for the clinical studies and analyzed with the scalable full spectral MC analysis were used to evaluate the ratiometric analysis. The averaged diffuse reflectance spectrum for each site from each study was analyzed with both the inverse full spectral MC analysis and the ratiometric analysis. Pearson correlation coefficients between the full spectral MC and ratiometric analysis extracted [THb] and SO₂ values were calculated for each clinical study. In the cervical study, patients referred from the Duke University Medical Center (DUMC) Colposcopy Clinic after abnormal Papanicolaou tests were recruited. A fiber optic probe was used to deliver and collect the diffuse reflectance (350-600 nm) from one to three visually abnormal sites immediately after colposcopic examination of the cervix with the application of 5% acetic acid. This was followed by an optical measurement on a coloposcopically normal site from the same patient. Optical measurements of colposcopically normal and abnormal sites were taken prior to biopsy to avoid confounding absorption due to superficial bleeding. Diffuse reflectance from 76 sites in 38 patients were normalized by a reflectance standard and interpolated prior to calculating the reflectance ratios. Reduced scattering coefficients, [THb] and SO₂ were also extracted from the same data using the inverse full spectral MC analysis.

For the head and neck cancer in vivo study, 42 enrolled patients had undergone panendoscopy with biopsy. After the consented patient was under general anesthesia, the optical probe was placed on at least two sites: a clinically suspicious site and a distant normal site with normal mucosa appearance whose location was contralaterally matched to the suspicious site. At least 5 diffuse reflectance spectra were measured for each site. The biopsies were obtained immediately after the probe was removed from the measured clinical suspicious sites. All measurements were calibrated to the reflectance standard measured on the day of the surgery. In this head and neck study, the utility of the physiological and morphological endpoints obtained via the quantitative diffuse reflectance spectroscopy technique was investigated for the classification of head and neck squamous cell carcinoma at the time of staging panendoscopy. Malignant and non-malignant tissues were initially stratified by diagnosis and further classified by anatomical and morphological groupings to determine the most effective approach to discriminate squamous cell carcinoma (SCC) from its benign counterparts.

In the breast cancer study, thirty-five patients undergoing either a modified radical mastectomy or partial mastectomy for invasive and noninvasive breast malignancies were recruited. The surgeon first located the lesion under ultrasound guidance; then, either a 10-gauge or 14-gauge biopsy needle coaxial cannula was guided through a small incision in the skin into the region of interest. A diffuse reflectance measurement (350-600 nm) was collected at a distance of 2 mm past the cannula with a fiber-optic probe after the removal of the needle and residual blood in the field. The optical probe was then retracted, and a biopsy needle was inserted through the cannula and a biopsy sample was removed. This resulted in the removal of a typically 20-mm-long cylinder of tissue, the proximal end of which corresponded to the volume optically measured by the probe. Tissue reflectance spectra from biopsies were normalized by the diffuse reflectance measured from an integrating sphere (Labsphere. Inc. North Sutton. N.H.) at the same day of the surgery for each patient. Biopsy samples were further processed through standard histologic procedures for pathological information.

To compare the classification performances of the full spectral MC and ratiometric analyses, w the area under the receiver operating curves (AUC) calculated from the logistic regression models built were compared based on the optical biomarkers extracted from the two analyses. The AUC may be more representative for the classification performance since the AUC is generated from various cut-off criteria. Since the full spectral MC model is able to extract optical biomarkers rather than just [THb] and SO₂, μₛ′ extracted with the for the full spectral MC model to build the logistic regression model for the cervix, breast and the head and neck groups. Beta-carotene concentrations extracted with the full spectral MC model were also included to build the logistic regression model for the breast group. The extracted [THb], μs′ and the beta-carotene concentrations were log transformed before building the logistic regression model. The p values were computed based on a suitable method for comparing the ROC curves. All logistic regression models and the p values were computed with the SAS software (SAS Institute Inc., Cary, N.C., USA).

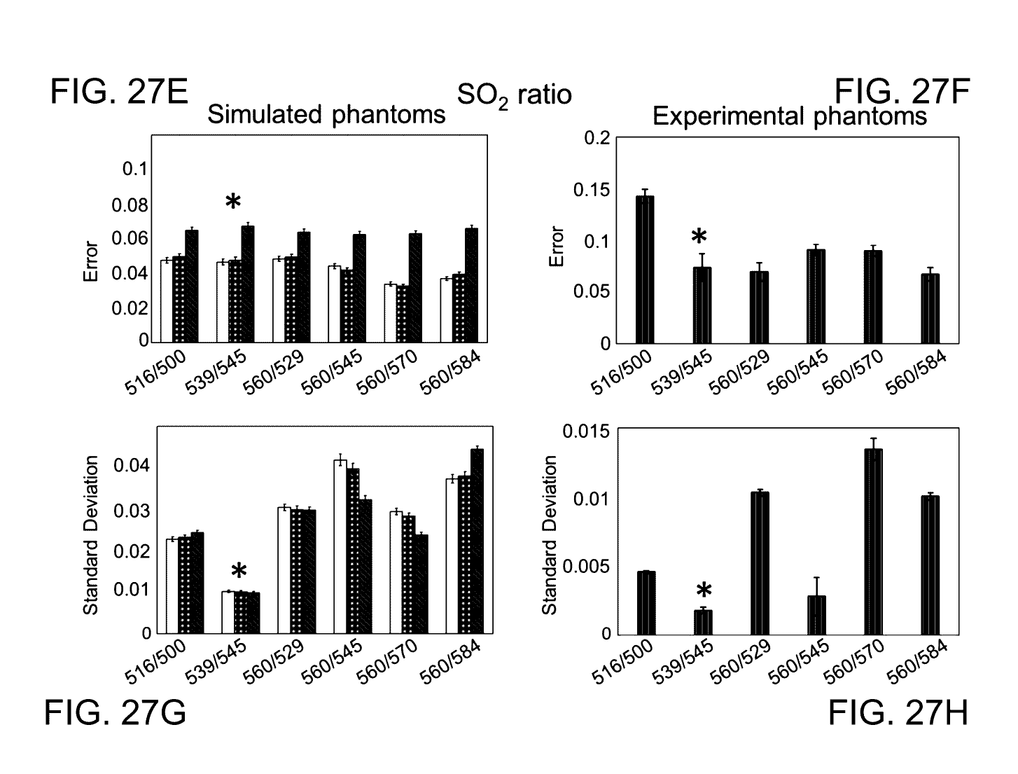

The accuracy of the 10 isosbestic wavelength-pairs to extract [THb] was evaluated in both simulated and experimental phantoms. Errors in extracted [THb] for each ratio were calculated. Next, the standard deviation of each ratio for changes in tissue scattering and SO₂ was computed using only the simulated data. The 10 ratios were then ranked using both the standard deviations and the errors. The best ratio should be able to accurately extract [THb] with low sensitivity to both tissue scattering and SO₂. A total of 25 wavelength-pairs were available for the calculation of SO₂. The accuracy of these wavelength-pairs to determine SO2 was also ranked using an identical metric as was used for [THb]. Again, the best ratio should be able to accurately extract SO₂ with low sensitivity to tissue scattering. FIGS. 27A-27H show results for the simulated phantoms and the experimental phantoms. Errors and ratio standard deviation of [THb] ratios and SO₂ ratios from simulated phantoms and experimental phantoms. The top 6 ratios as defined by the lowest errors are shown. FIGS. 27A and 27B show errors of the top 6 [THb] ratios in simulated data and experimental data. 584/545 has the lowest errors in both simulated phantom data and experimental phantom data. FIGS. 27C and 27D show standard deviations of the top 6 [THb] ratios in the simulated data and the experimental data. 570/545, 584/545, and 584/570 have low standard deviation in both data sets. FIGS. 27E and 27F show errors of the top 6 SO₂ ratios in the simulated and experimental data. The errors are comparable for these ratios except for 516/500, which has higher errors in the experimental data. FIGS. 27G and 27H show standard deviations of the top 6 SO₂ ratios in the simulated data and the experimental data. 539/545 has the lowest standard deviation in both data sets. The best ratios for extracting [THb] or SO₂ are marked with asterisk (*). FIGS. 27A and 27B show 6 ratios with the lowest errors to extract [THb] in the simulated and experimental datasets, respectively. FIGS. 27C and 27D show the standard deviation of the [THb] ratios for various SO₂ and scattering levels in the simulated and experimental data, respectively. FIGS. 27E and 27H show similar data for SO₂. For [THb] ratios, 584/545 has the lowest average errors for each band pass in both simulated and experimental phantoms. The standard deviation of 584/545 was the third lowest for each band pass in simulated data and the second lowest for each band pass in experimental phantoms. This means that 584/545 can extract [THb] with relatively small errors, and it is relatively insensitive to the scattering or SO₂. The average errors are comparable in both simulations and in experimental phantoms for the top 6 SO₂ ratios with the exception of 516/500, which has higher errors in the experimental phantom. 539/545 has the lowest average ratio and standard deviation in both simulation and experimental phantoms. Thus, 584/545 and 539/545 were chosen as [THb] and SO₂ ratios for further testing.

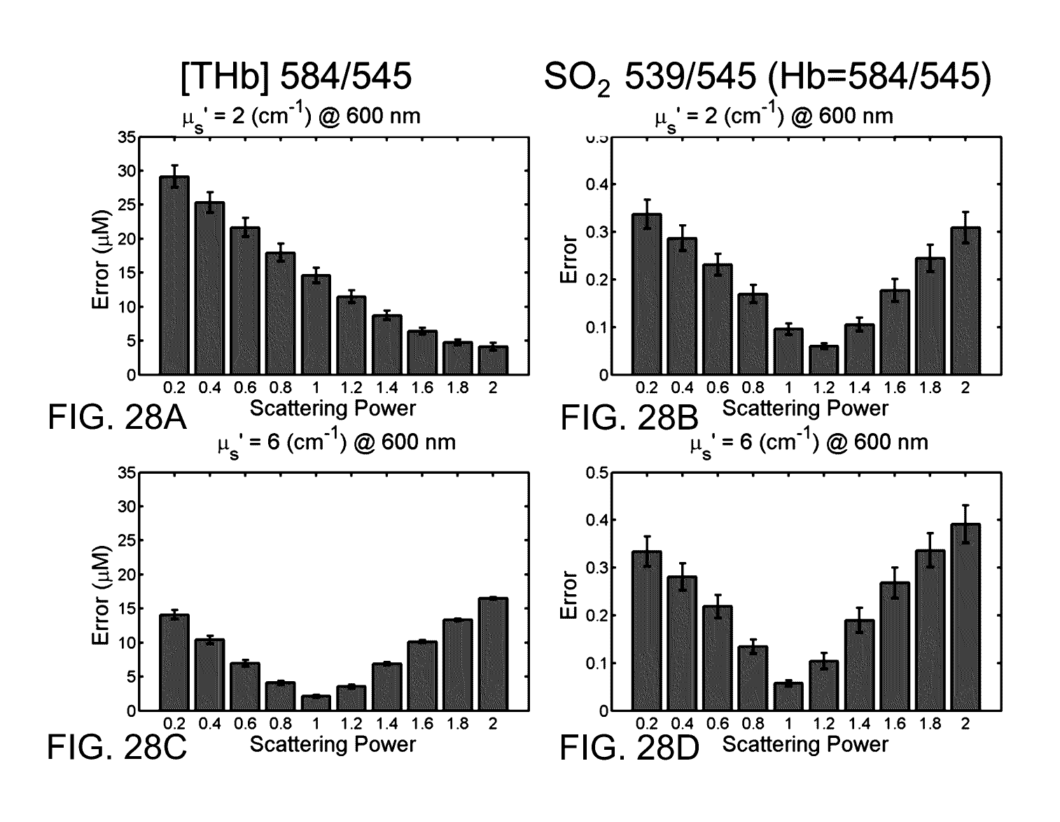

FIGS. 28A-28H show the absolute errors of the extracted [THb] and SO₂ for the best [THb] and SO₂ ratios when using the scatter power model. The accuracies for extracting [THb] and SO₂ varied with scattering power. In the obtained data, the average and the standard deviation of the scattering power for head and neck, cervix and breast tissues are 0.6260.12, 0.5560.27 and 0.5060.16 respectively. More particularly, FIGS. 28A, 28C, and 28E show absolute errors for extracting the [THb] of the simulated reflectance spectra with 584/545 when the scattering power varied from 0.2 to 2 for different scattering levels. FIGS. 28B, 28D, and 28F show absolute errors for extracting the SO₂ of the simulated reflectance spectra with 539/545 when the scattering power varied from 0.2 to 2 for different scattering levels. FIG. 28G show averaged errors from FIGS. 28A, 28C, and 28E. FIG. 28H shows average errors from FIGS. 28B, 28D, and 28F. Error bars represent the standard errors.

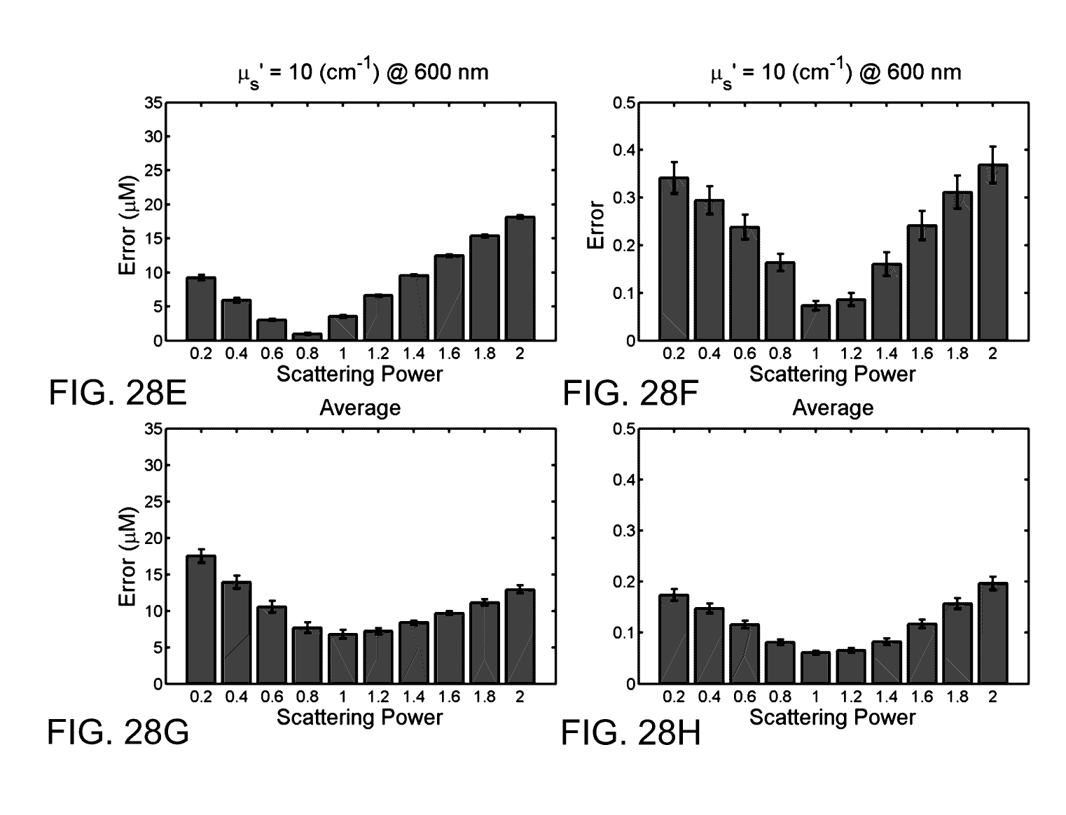

FIGS. 29A-29C show the comparison of the computational time, the mean error in [THb] extraction, and the mean error in SO₂ extraction using the scalable full spectral MC analysis and the ratiometric analysis. More particularly, FIG. 29A shows elapsed time of extracting 100 MC simulated phantoms for the scalable inverse MC model and the ratiometric analysis. FIG. 29B shows absolute [THb] error. FIG. 29C shows SO₂ errors for MC and ratiometric analysis. These data were generated using 100 simulated diffuse reflectance spectra. [THb] was extracted using the ratiometric analysis with the ratio computed between 584 nm and 545 nm. The extracted [THb] value from the ratiometric analysis was then used to determine the look-up coefficients to calculate the SO₂ using the 539 nm/545 nm ratio. As shown in FIG. 29A, the ratiometric analysis is over 4000 times more computationally efficient compared to the full spectral MC analysis. FIGS. 29B and 29C show the mean error for [THb] extraction and SO₂ extraction using the full spectral MC analysis and the ratiometric analysis. The mean errors were 0.24 mM and 3.94 mM for [THb] extraction, while the errors were 0.004 and 0.23 for SO₂ values for the MC analysis and the ratiometric analysis, respectively.

Correlation coefficients were computed between the optical endpoints extracted using both analyses for each tissue group in each of the three clinical studies. Table 5 below summarizes the Pearson correlation coefficients between the full spectral MC analysis and the ratiometric analysis for [THb] and SO₂ of each tissue group in the cervical pre-cancer, head and neck squamous cell carcinoma, and breast cancer studies.

| | | [THb] | | SO₂ | |
|---|---|---|---|---|---|
| Study | | r | P | r | P |
| Cervix | All Tissues | 0.69 | <0.01 | 0.43 | <0.01 |
| | Normal | 0.66 | <0.01 | 0.34 | 0.02 |
| | CIN1 | 0.62 | 0.01 | 0.5 | 0.05 |
| | CIN2+ | 0.76 | <0.01 | 0.46 | 0.13 |
| Head and neck | All Tissues | 0.92 | <0.01 | 0.87 | <0.01 |
| | Glottic | 0.97 | <0.01 | 0.91 | <0.01 |
| | Lymphoid | 0.72 | <0.01 | 0.85 | <0.01 |
| | Mucosal | 0.92 | <0.01 | 0.87 | <0.01 |
| Breast | All Tissues | 0.77 | <0.01 | 0.71 | <0.01 |
| | Tumor | 0.85 | <0.01 | 0.63 | <0.01 |
| | Benign | 0.71 | <0.01 | 0.56 | <0.01 |
| | Adipose | 0.82 | <0.01 | 0.48 | <0.01 |

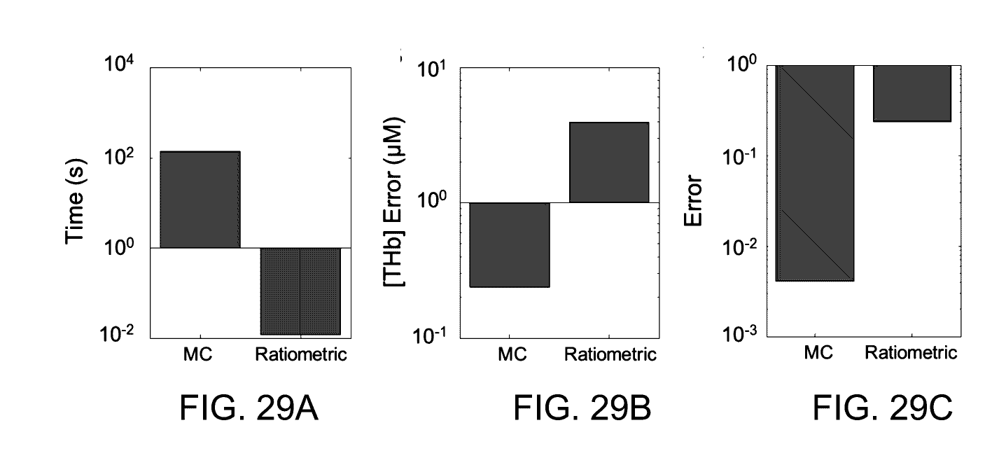

The normal samples in the breast cancer study were further classified into the benign and adipose group, depending on the adipose percentage of the normal sample. The overall correlation coefficient for each study was also computed when all samples in each study were used.

[THb] was extracted using the inverse full spectral MC analysis from a total of 76 samples from 38 patients, as published previously. The samples were classified as normal, low-grade cervical intraepithelial neoplasia (CIN 1) and high-grade cervical intraepithelial neoplasia (CIN 2+). FIGS. 30A and 30B show results for the in vivo cervix study. FIG. 30A shows boxplots for the full spectral MC extracted [THb] for the three tissue groups. FIG. 30B shows boxplots for [THb] extracted using the ratiometric analysis for the three tissue groups. To compare with the previous results extracted by the full spectral MC analysis, a log transformation was applied to the ratiometrically extracted [THb]. [THb] determined using both analyses was statistically higher in CIN2+ tissues (p,0.01) compared to normal and CIN1 samples. No statistical differences were found when comparing the SO₂ of different tissue groups with the full spectral MC analysis or the ratiometric analysis. The p-values were derived from the unpaired two-sided student t-tests for consistency with the previously published data.

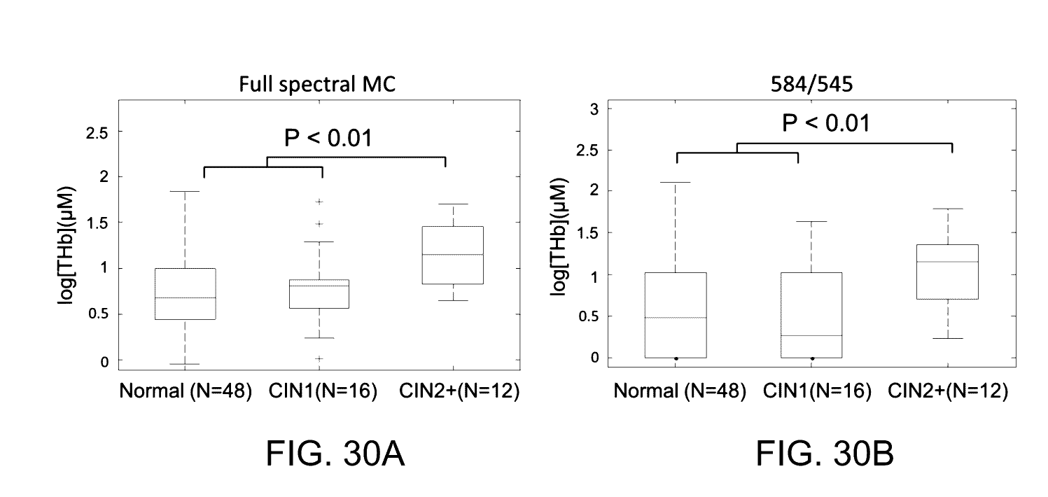

FIGS. 31A and 31B show boxplots for SO₂ values extracted with full spectral MC analysis and the ratiometric analysis, across all measured tumor and normal sites in head and neck squamous cell carcinoma patients. The samples were separated into 3 groups (glottic, lymphoid and mucosal) based on morphological location of each measurement site. Wilcoxon rank-sum tests were used to establish differences between the extracted SO₂ values in the normal and SCC sites, for each tissue group. The extracted SO₂ was significantly different between SCC and normal samples for the glottic, lymphoid and mucosal tissue groups when extracted using both the full spectral MC analysis (p=0.03, p,0.01 and p=0.01 respectively) and the ratiometric analysis. SO₂ values extracted using the ratiometric analysis (p,0.01 for the 3 groups) showed similar differences between the SCC and normal samples for these tissue groups.

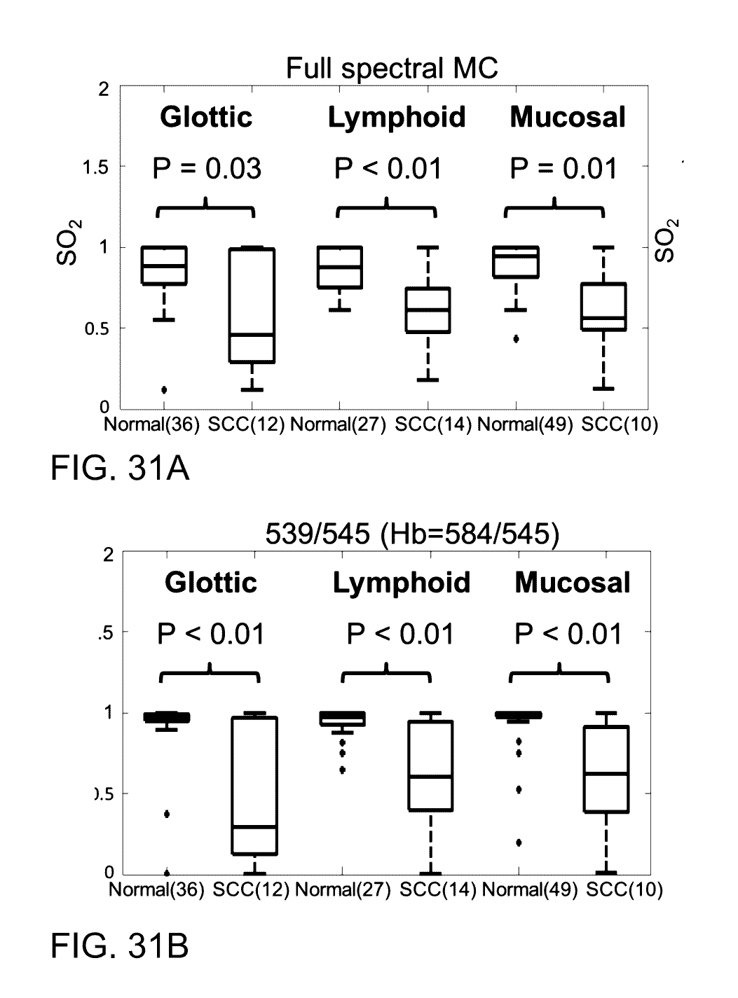

FIGS. 32A-32D show boxplots for the inverse full spectral MC or the ratiometrically extracted SO₂ of malignant and normal breast tissues. The boxplots of FIGS. 32A and 32B were extracted with full spectral Monte Carlo analysis and the ratiometric analysis, respectively. The normal samples were reclassified into a benign group if the fat content of the tissue biopsy was less than 50% or into the adipose group if the fat content in the biopsy was greater than 50%. FIG. 32A shows boxplots for the full spectral MC extracted SO₂ of the tumor and benign tissues whereas FIG. 32B shows boxplots for the ratiometrically extracted SO₂ of tumor and benign tissue from the in vivo breast study. FIGS. 32C and 32D also show boxplots for the SO₂ of the tumor and adipose tissues extracted with both analyses. Wilcoxon ranksum tests were performed to test the statistical significance of the extracted SO₂ between the tumor samples and normal (both benign and adipose) tissues for both full spectral MC analysis and ratiometric analysis. The extracted SO₂ of the normal samples were significantly higher than the tumor samples (p,0.01) for both the ratiometric analysis and the full spectral MC analysis.

The combinations of the optical biomarkers used for building the logistic regression models and the area under the receiver operating curve (ROC) are summarized in Table 6 below.

| | | | | |
|---|---|---|---|---|
| Full spectral MC | | Ratiometric | | p |
| optical biomarkers | AUC | optical biomarkers | AUC | value |

Breast

| | | | | |
|---|---|---|---|---|
| SO₂, log([THb]) | 0.83 | SO₂, log([THb]) | 0.79 | 0.42 |
| SO₂, log([THb]), | 0.85 | SO₂, log([THb]) | 0.79 | 0.26 |
| log(μₛ′) | | | | |
| SO₂, log([THb]), | 0.85 | SO₂, log([THb]) | 0.79 | 0.24 |
| log(μₛ′), | | | | |
| log(β-carotene) | | | | |

Cervix

| | | | | |
|---|---|---|---|---|
| SO₂, log([THb]) | 0.76 | SO₂, log([THb]) | 0.72 | 0.51 |
| SO₂, log([THb]), | 0.77 | SO₂, log([THb]) | 0.72 | 0.54 |
| log(μₛ′) | | | | |

Head and neck (Glottic)

| | | | | |
|---|---|---|---|---|
| SO₂, log([THb]) | 0.79 | SO₂, log([THb]) | 0.76 | 0.69 |
| SO₂, log([THb]), | 0.83 | SO₂, log([THb]) | 0.76 | 0.48 |
| log(μₛ′) | | | | |

Head and neck (Lymphoid)

| | | | | |
|---|---|---|---|---|
| SO₂, log([THb]) | 0.90 | SO₂, log([THb]) | 0.85 | 0.32 |
| SO₂, log([THb]), | 0.89 | SO₂, log([THb]) | 0.85 | 0.40 |
| log(μₛ′) | | | | |

Head and neck (Mucosal)

| | | | | |
|---|---|---|---|---|
| SO₂, log([THb]) | 0.81 | SO₂, log([THb]) | 0.86 | 0.13 |
| SO₂, log([THb]), | 0.83 | SO₂, log([THb]) | 0.86 | 0.31 |
| log(μₛ′) | | | | |

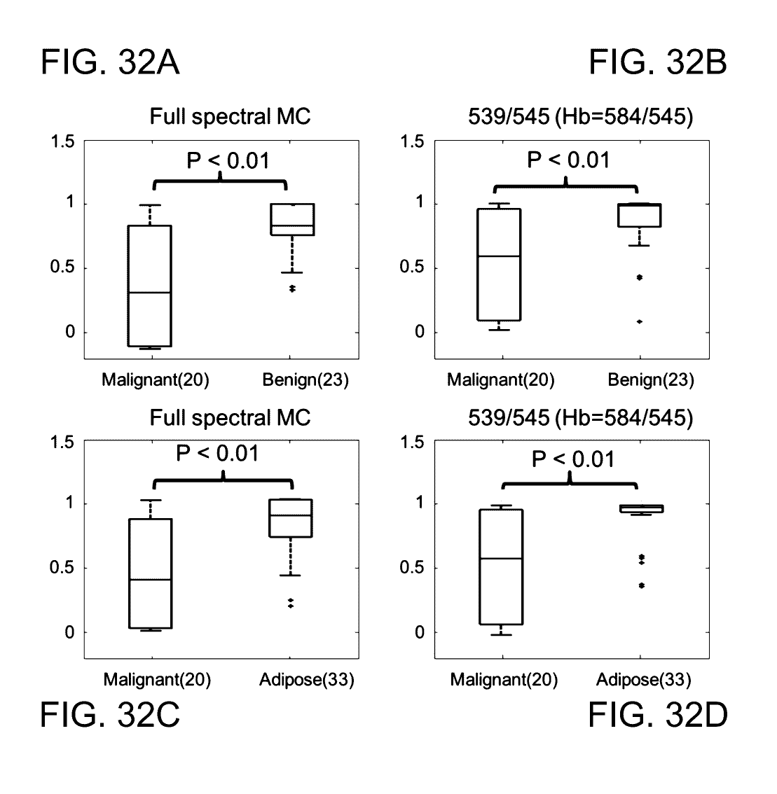

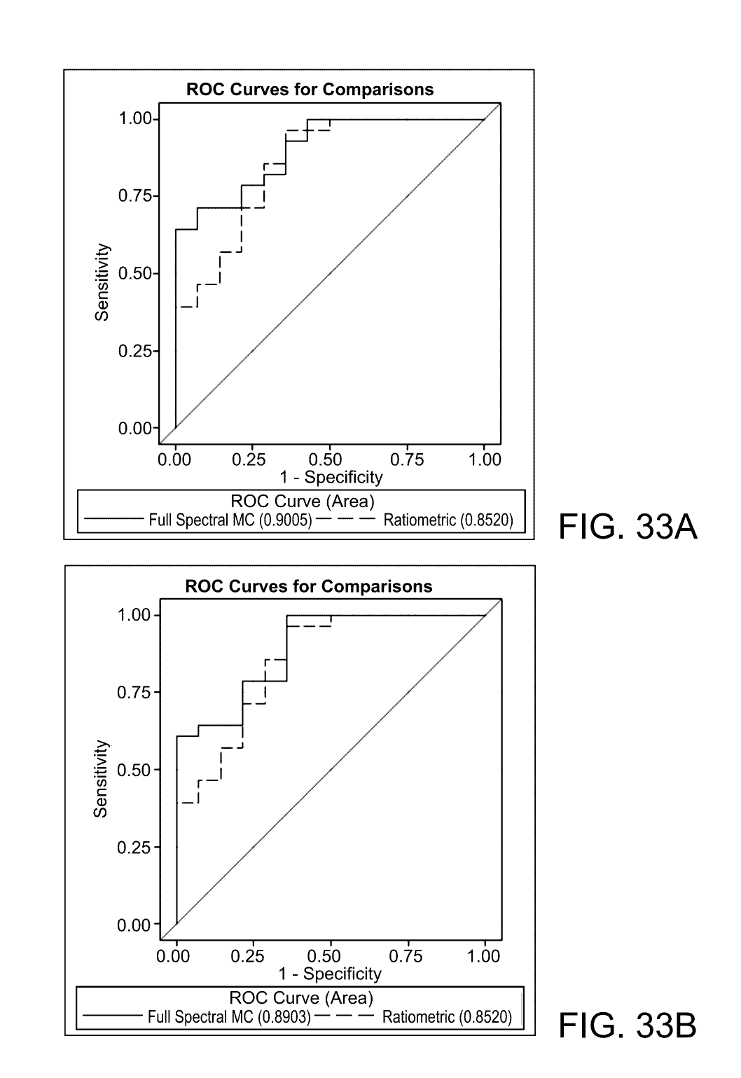

No significant p values were observed when comparing the AUC calculated between the two analyses. Representative ROC curves built based on the optical biomarkers extracted from the lymphoid tissues using the full spectral MC and the ratiometric analyses are also shown in FIGS. 33A and 33B. FIG. 33A shows a full spectral MC and the ratiometrically extracted SO₂, log([THb]) that were used for building the MC and the ratiometric logistic regression models respectively. FIG. 33B shows SO₂, log([THb]), log(μₛ′) used to build the logistic regression model for the full spectral MC analysis and the ratiometric ROC curve was built based on the SO₂ log([THb]). The full spectral MC ROC curve in FIG. 33A was built based on the SO₂ and the log([THb]) and the full spectral MC ROC curve in FIG. 33B was built based on SO₂, log([THb]) and log(ms′). Both ratiometric ROC curves in FIGS. 33A and 33B were built based on SO₂ and log([THb]).

A simple and fast analysis for quantitative extraction of [THb] and SO₂ of tissues is disclosed. The analysis may use a look-up table that allows conversion of the ratio of the diffuse reflectance at two selected wavelengths into [THb] and SO₂ values. This ratiometric analysis uses two isosbestic wavelengths for the calculation of [THb] and one isosbestic wavelength along with a wavelength where a local maximum difference in the extinction coefficients of deoxy- and oxy-hemoglobin exists for SO₂. A total of 10 wavelength-pairs were tested for extraction of the [THb] while 25 wavelength-pairs were tested for SO₂. The wavelength-pairs with the least dependence on tissue scattering were selected through rigorous tests on a total of 24805 spectra. The look-up tables may be used to translate the reflectance ratio into quantitative values were built for specific experimental probe-geometries and theoretically can be extended to any given source-detector configuration. Further, calibration using specific experimental phantoms ensured that the ratiometric analysis could directly be used on experimentally measured data. Once analytical equations for the ratiometric analysis were generated, extraction of [THb] and SO₂ values from experimentally measured diffuse reflectance was over 4000 times faster than the scalable inverse full spectral MC analysis with minimal loss in accuracy. Even though the ratiometric analysis is not expected be as accurate as the inverse full spectral MC analysis, the ratiometric analysis achieves similar contrast between malignant and the benign tissues in three different organ sites for a wide range of tissue vascularity and for tissues with multiple absorbers.

A prominent hemoglobin absorption feature (Soret band) occurred around 410-420 nm in the visible spectrum. However, the absorption peaks of hemoglobin were omitted around the 410-420 nm since most silicon-based detectors have lower sensitivities in this region. In order to detect the hemoglobin absorption around 410-420 nm, higher power light sources or more sensitive detectors may be required. In order to leverage relatively low priced light sources, the wavelengths were chosen from 500 nm to 600 nm (visible spectrum) in this example.

The purpose of the bandpass simulations was to understand if the best [THb] or the SO₂ ratios would change for the different systems used. Results show that 584/545 and 539/545 are the best ratios for the simulated results with three different bandpass values. Both 584/545 and 539/545 can extract [THb] or SO₂ with low errors and both ratios have low sensitivity to scattering. Although different systems might have different bandpasses, the relative rankings of the [THb] ratios and SO₂ ratios for error and the sensitivity to scattering remain the same. The clinical data has three different bandpasses. The band passes were 1.5 nm and 1.9 nm for the head and neck and breast data, respectively. The bandpasses were 1.9 nm or 3.5 nm for the cervical data. The extracted data with the ratiometric analysis show good agreement with the full spectral MC extracted values. In addition, the simulated [THb] results in FIGS. 27A and 27B are consistent except that 570/545 has higher errors in the experimental data. It is expected that the 74415 (24805 spectra*3 different bandpass values) MC-simulated spectra can account for a wide range of optical properties and thus, is more comprehensive than the experimental data.

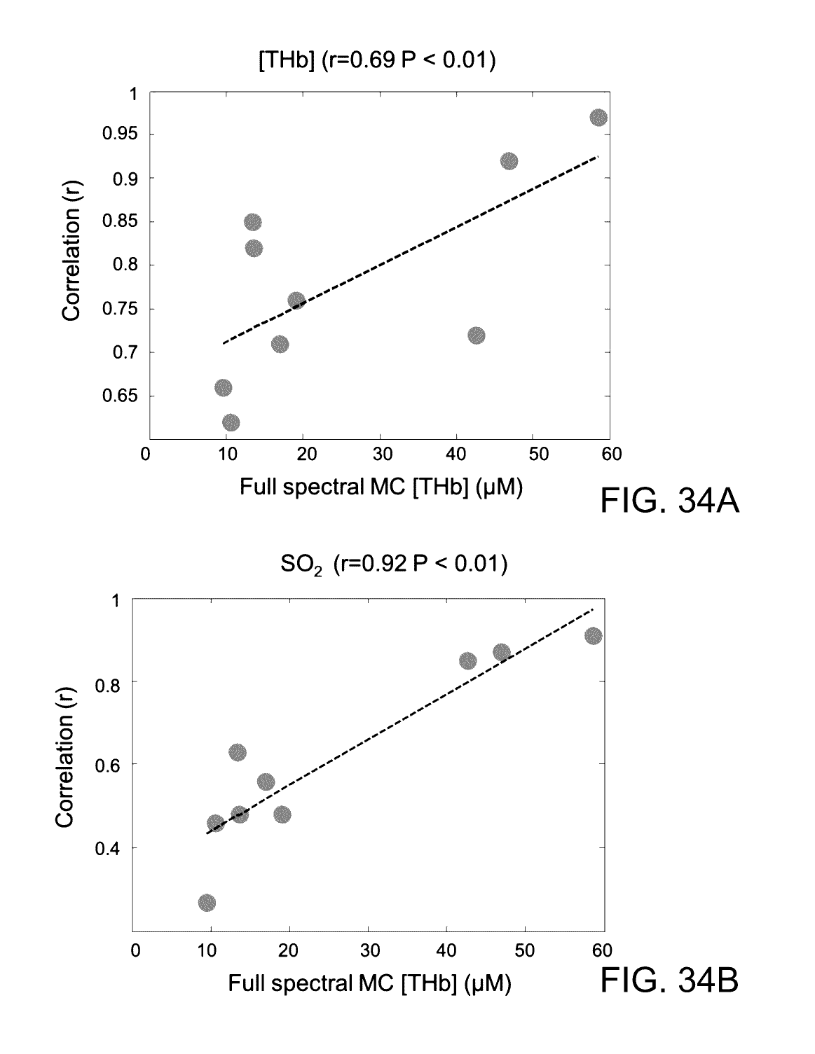

The sensitivities of the ratiometric analysis to the scattering power were tested since the scattering power is likely to change in the real tissues. As can be seen in FIGS. 28A-28H, the accuracies varied as the scattering power has changed. Although the ratiometric analysis is less accurate when the scattering power varies than when the scattering power is a constant, the contrast between malignant and non-malignant tissues in breast and head and neck or the contrast between the low-grade and the high-grade cervical tissues is still preserved. In addition, an analysis found a high degree of correlation in the extracted [THb] and SO₂ values between the ratiometric analysis and the inverse full spectral MC analysis. These correlations were especially high for measurements in head and neck tissues. Correlations between the extracted [THb] and SO₂ in cervical tissues were the lowest, relative to head and neck or breast tissues. These effects might be due to the fact that the [THb] was typically much higher in the head and neck and breast studies, relative to the cervical study (the averaged full spectral MC extracted [THb] for head and neck, breast and cervical tissues were, 57.8 mM, 14.2 mM and 5.9 mM respectively). In other words, correlations between the ratiometrically and the full spectral MC extracted [THb] or SO₂ are positively correlated to the full spectral MC extracted [THb]. This can be seen in FIGS. 34A and 34B, which show the scatter plot for the average MC extracted [THb] for the 9 tissue groups in Table 5 versus the correlation coefficients between the full spectral MC extracted and ratiometrically-extracted [THb] and SO₂. Because hemoglobin has very high extinction coefficients in the UV spectral-range relative to the visible, using wavelengths in the UV range could provide increased dynamic-range for sensing hemoglobin. This reasoning supports another study, where the ratiometric technique for extraction of [THb] was superior for the 545/390, 452/390 and 529/390 ratios, relative to the 584/545 wavelength pair used here.

Although the ratiometric analysis was developed by assuming that hemoglobin was the primary absorber in tissue, the experimental measurements on human tissue can be influenced by absorbers other than hemoglobin. However, SO₂ and [THb] are the hallmarks of carcinogenesis and represent the features of a growing tumor. This has been published on widely and is useful in diagnostics and therapeutics. For example, neovascularization increases with the development of cancer, and tumor hypoxia occurs as tumors outstrip their blood supply. Thus, being able to measure these endpoints with an optical technology that is optimized for speed and cost will have applications in early detection, diagnostics and response to therapy. Although some tissues may have multiple absorbers in addition to Hb, the classification performances were not significantly affected when using only [THb] and SO₂ as parameters (in cervix and head & neck only). Further, optical technologies have a significant potential to have an impact in global health. The ratiometric analysis still worked well in breast tissue, where beta-carotene is a known absorber in the wavelength range used. The presence of beta-carotene may be one reason why a slightly lower correlation coefficients between the ratiometric and full spectral MC analysis was obtained in the breast study, relative to the head and neck study. Overall, in all of the clinical studies, the [THb] extracted from the ratiometric analysis were better correlated to full spectral MC values, in comparison to the SO₂ values. The effect of beta-carotene is more obvious in the SO2 estimation than in the [THb] estimation. This may possibly be due to the absorption of beta-carotene being 8.5 times lower in the 550-600 nm compared to 500-550 nm. However, despite the lower correlation for the SO₂ estimation in the breast tissues, the ratiometric analysis is still able to preserve the contrast between the malignant and non-malignant breast tissues observed with the results using the full spectral MC analysis.

Herein, it is shown the potential utility of the ratiometric analysis for diffuse reflectance imaging. Since the ratiometric analysis only involves wavelengths at 539, 545 and 584 nm, this analysis can be incorporated into any system with the use of a simple white LED and appropriate bandpass filters as disclosed by the examples provided herein. With appropriate optimization for wavelength and illumination and collection geometries, the ratiometric analysis might be applied to a variety of spectral imaging systems. For example, this analysis can be incorporated into previously developed fiber-less technology, where a Xenon lamp and light filters are used to illuminate the tissue at different wavelengths of light. The illumination light was delivered through free space with a quartz light delivery tube. A custom photodiode array is in contact with the tissue to directly measure diffuse reflectance from a large area of tissue. With proper modifications of this system and combined with the ratiometric analysis, real-time [THb] and SO₂ imaging is possible.

A rapid analytical ratiometric analysis for determining [THb] and SO₂ in head and neck, cervical, and breast tissues was presented. This analysis is non-invasive, label-free, quantitative, and fast. The ratiometric analysis requires the diffuse reflectance only from three selected wavelengths to calculate both [THb] and SO₂. Thus, the system design can be simple, portable, and potentially useful for global health applications. The fast computation speed allows near real-time [THb] and SO₂ mapping of tissue. This can provide important physiological information for many clinical applications, from cancer screening to diagnostics to treatment.

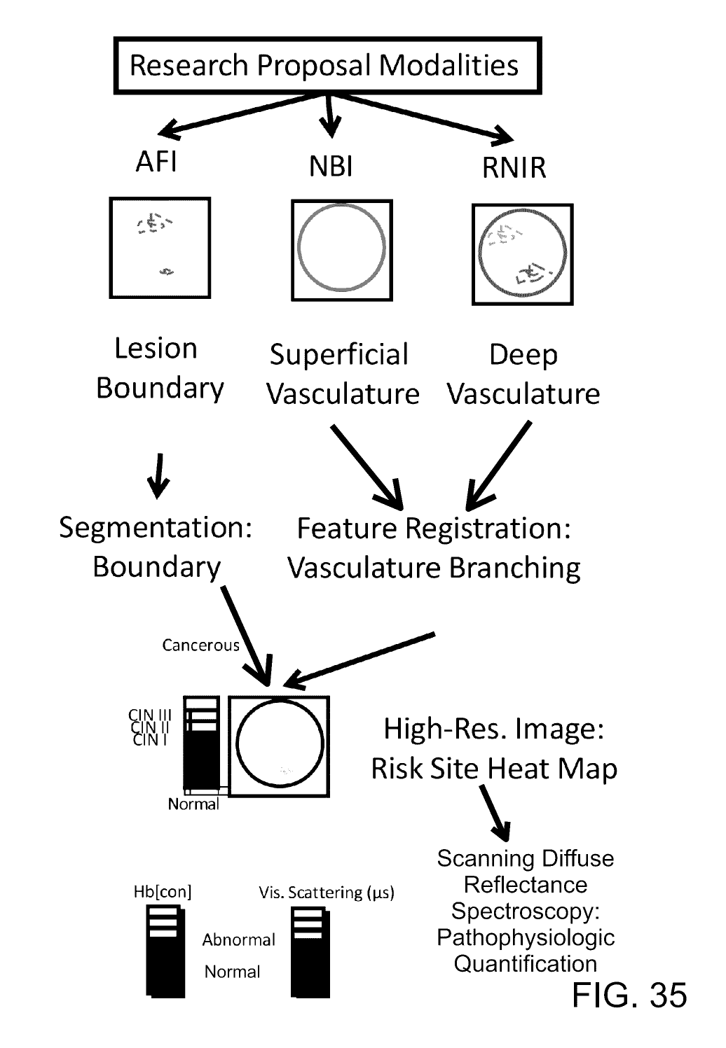

FIG. 35 illustrates a flow diagram for an example colposcopy method in accordance with embodiments of the present disclosure. Referring to FIG. 35, the multi-modal approach that may be implemented after white-field imaging may then be sequentially imaged with auto-fluorescent (e.g., UV or near UV LED source) to obtain information about the collagen and metabolic content of the cervical tissue or other bodily tissue which can be processed with a suitable segmentation technique to stratify and identify boundaries of suspicious regions, followed by narrow band imaging (with narrow blue and green wavelengths) to obtained detailed information about the superficial vasculature of the cervix and followed by near infrared to infrared imaging to obtain deeper vasculature of the cervix where both sets can be processed by feature registration and Gabor wavelet filtering to gather more detailed vascular information and be extracted to gather ratiometric parameters.

The present disclosure may be a system, a method, and/or a computer program product. The computer program product may include a computer readable storage medium (or media) having computer readable program instructions thereon for causing a processor to carry out aspects of the present disclosure.

The computer readable storage medium can be a tangible device that can retain and store instructions for use by an instruction execution device. The computer readable storage medium may be, for example, but is not limited to, an electronic storage device, a magnetic storage device, an optical storage device, an electromagnetic storage device, a semiconductor storage device, or any suitable combination of the foregoing. A non-exhaustive list of more specific examples of the computer readable storage medium includes the following: a portable computer diskette, a hard disk, a random access memory (RAM), a read-only memory (ROM), an erasable programmable read-only memory (EPROM or Flash memory), a static random access memory (SRAM), a portable compact disc read-only memory (CD-ROM), a digital versatile disk (DVD), a memory stick, a floppy disk, a mechanically encoded device such as punch-cards or raised structures in a groove having instructions recorded thereon, and any suitable combination of the foregoing. A computer readable storage medium, as used herein, is not to be construed as being transitory signals per se, such as radio waves or other freely propagating electromagnetic waves, electromagnetic waves propagating through a waveguide or other transmission media (e.g., light pulses passing through a fiber-optic cable), or electrical signals transmitted through a wire.

Computer readable program instructions described herein can be downloaded to respective computing/processing devices from a computer readable storage medium or to an external computer or external storage device via a network, for example, the Internet, a local area network, a wide area network and/or a wireless network. The network may comprise copper transmission cables, optical transmission fibers, wireless transmission, routers, firewalls, switches, gateway computers and/or edge servers. A network adapter card or network interface in each computing/processing device receives computer readable program instructions from the network and forwards the computer readable program instructions for storage in a computer readable storage medium within the respective computing/processing device.

Computer readable program instructions for carrying out operations of the present disclosure may be assembler instructions, instruction-set-architecture (ISA) instructions, machine instructions, machine dependent instructions, microcode, firmware instructions, state-setting data, or either source code or object code written in any combination of one or more programming languages, including an object oriented programming language such as Java, Smalltalk, C++ or the like, and conventional procedural programming languages, such as the “C” programming language or similar programming languages. The computer readable program instructions may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider). In some embodiments, electronic circuitry including, for example, programmable logic circuitry, field-programmable gate arrays (FPGA), or programmable logic arrays (PLA) may execute the computer readable program instructions by utilizing state information of the computer readable program instructions to personalize the electronic circuitry, in order to perform aspects of the present disclosure.

Aspects of the present disclosure are described herein with reference to flowchart illustrations and/or block diagrams of methods, apparatus (systems), and computer program products according to embodiments of the disclosure. It will be understood that each block of the flowchart illustrations and/or block diagrams, and combinations of blocks in the flowchart illustrations and/or block diagrams, can be implemented by computer readable program instructions.

These computer readable program instructions may be provided to a processor of a general purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks. These computer readable program instructions may also be stored in a computer readable storage medium that can direct a computer, a programmable data processing apparatus, and/or other devices to function in a particular manner, such that the computer readable storage medium having instructions stored therein comprises an article of manufacture including instructions which implement aspects of the function/act specified in the flowchart and/or block diagram block or blocks.

The computer readable program instructions may also be loaded onto a computer, other programmable data processing apparatus, or other device to cause a series of operational steps to be performed on the computer, other programmable apparatus or other device to produce a computer implemented process, such that the instructions which execute on the computer, other programmable apparatus, or other device implement the functions/acts specified in the flowchart and/or block diagram block or blocks.

The flowchart and block diagrams in the Figures illustrate the architecture, functionality, and operation of possible implementations of systems, methods, and computer program products according to various embodiments of the present disclosure. In this regard, each block in the flowchart or block diagrams may represent a module, segment, or portion of instructions, which comprises one or more executable instructions for implementing the specified logical function(s). In some alternative implementations, the functions noted in the block may occur out of the order noted in the figures. For example, two blocks shown in succession may, in fact, be executed substantially concurrently, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts or carry out combinations of special purpose hardware and computer instructions.

The terminology used herein is for the purpose of describing particular embodiments only and is not intended to be limiting of the disclosure. As used herein, the singular forms “a,” “an” and “the” are intended to include the plural forms as well, unless the context clearly indicates otherwise. It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof.

The corresponding structures, materials, acts, and equivalents of all means or step plus function elements in the claims below are intended to include any structure, material, or act for performing the function in combination with other claimed elements as specifically claimed. The description of the present disclosure has been presented for purposes of illustration and description, but is not intended to be exhaustive or limited to the present disclosure in the form disclosed. Many modifications and variations will be apparent to those of ordinary skill in the art without departing from the scope and spirit of the present disclosure. The embodiment was chosen and described in order to best explain the principles of the present disclosure and the practical application, and to enable others of ordinary skill in the art to understand the present disclosure for various embodiments with various modifications as are suited to the particular use contemplated.

The descriptions of the various embodiments of the present disclosure have been presented for purposes of illustration, but are not intended to be exhaustive or limited to the embodiments disclosed. Many modifications and variations will be apparent to those of ordinary skill in the art without departing from the scope and spirit of the described embodiments. The terminology used herein was chosen to best explain the principles of the embodiments, the practical application or technical improvement over technologies found in the marketplace, or to enable others of ordinary skill in the art to understand the embodiments disclosed herein.

What is claimed is:

1. A colposcope comprising:
an elongate body having a distal end, a proximate end, and an axis extending between the distal end and the proximate end;
a balloon attached to the elongate body and configured to be inflated to expand in a direction away from the axis of the elongate body;
an image capture device attached to the distal end of the elongate body and positioned to capture images of an area outside the elongate body; and
at least one light emitter attached to the distal end of the elongate body and positioned to generate and direct light towards the area outside of the elongate body.

2. The colposcope of claim 1, wherein the elongate body is tubular shaped.

3. The colposcope of claim 1, wherein the elongate body defines an interior space,
wherein colposcope comprises:
an interface at the proximal end of the elongate body; and
at least one cable being positioned in the interior space and operatively connecting the interface with the at least one image capture device and the at least one light emitter.

4. The colposcope of claim 3, wherein the interface is configured to be connected to an electronic device for powering and communicating with the image capture device and the at least one light emitter.

5. The colposcope of claim 1, wherein the at least one light emitter comprises a plurality of light emitting diodes (LEDs).

6. The colposcope of claim 5, wherein the LEDs are each configured to generate one of white light, blue light, and green light.

7. The colposcope of claim 5, wherein the LEDs comprise at least one LED configured to generate white light, at least one LED configured to generate blue light, and at least one LED configured to generate green light.

8. The colposcope of claim 1, the at least one light emitter comprises a plurality of light emitters are positioned substantially around the image capture device.

9. The colposcope of claim 1, further comprising at least one filter positioned to intercept light generated by the at least one light emitter for filtering the light.

10. The colposcope of claim 9, wherein the filter is a polarizer.

11. The colposcope of claim 1, further comprising at least one filter positioned to intercept light received by the image capture device for filtering the light.

12. The colposcope of claim 11, wherein the filter is a polarizer.

13. A colposcope comprising:
an elongate body having a distal end, a proximate end, and an axis extending between the distal end and the proximate end;
a cavity expander attached to the elongate body and having at least one member configured to be controllably positioned between a first position and a second position, wherein the at least one member is positioned a distance further from the axis of the elongate body when in the second position than when in the first position;
an image capture device attached to the distal end of the elongate body and positioned to capture images of an area outside the elongate body; and
at least one light emitter attached to the distal end of the elongate body and positioned to generate and direct light towards the area outside of the elongate body.

14. The colposcope of claim 13, wherein the elongate body is tubular shaped.

15. The colposcope of claim 13, wherein the elongate body defines an interior space,
wherein colposcope comprises:
an interface at the proximal end of the elongate body; and
at least one cable being positioned in the interior space and operatively connecting the interface with the at least one image capture device and the at least one light emitter.

16. The colposcope of claim 15, wherein the interface is configured to be connected to an electronic device for powering and communicating with the image capture device and the at least one light emitter.

17. The colposcope of claim 13, wherein the at least one light emitter comprises a plurality of light emitting diodes (LEDs).

18. The colposcope of claim 17, wherein the LEDs are each configured to generate one of white light, blue light, and green light.

19. The colposcope of claim 17, wherein the LEDs comprise at least one LED configured to generate white light, at least one LED configured to generate blue light, and at least one LED configured to generate green light.

20. The colposcope of claim 13, the at least one light emitter comprises a plurality of light emitters are positioned substantially around the image capture device.

21. The colposcope of claim 13, further comprising at least one filter positioned to intercept light generated by the at least one light emitter for filtering the light.

22. The colposcope of claim 21, wherein the filter is a polarizer.

23. The colposcope of claim 13, further comprising at least one filter positioned to intercept light received by the image capture device for filtering the light.

24. The colposcope of claim 23, wherein the filter is a polarizer.

25. A method comprising:
applying light to tissue;
capturing the light reflected from the tissue;
determining a plurality of reflectance ratios based on the captured light for determining hemoglobin concentration and oxygen saturation of the tissue; and
applying a segmentation technique to determine a boundary of a suspicious region of the tissue.

26. The method of claim 25, wherein applying light comprises applying light of one or more of the green spectrum, the blue spectrum, and the white spectrum.

27. The method of claim 25, wherein applying light comprises applying light of one or more of the following wavelengths about 500 nanometers (nm), about 529 nm, about 545 nm, about 570 nm, about 584 nm, about 516 nm, about 539 nm, about 560 nm, about 577 nm, and about 593 nm.

28. The method of claim 25, wherein applying light comprises applying light to cervix tissue.

29. The method of claim 25, further comprising displaying suspicious region of the tissue.

30. The method of claim 25, further comprising using a colposcope for applying the light to tissue, and for capturing the light reflected from the tissue.

31. The method of claim 30, wherein the colposcope comprises:
an elongate body having a distal end, a proximate end, and an axis extending between the distal end and the proximate end;
a balloon attached to the elongate body and configured to be inflated to expand in a direction away from the axis of the elongate body;
an image capture device attached to the distal end of the elongate body and positioned to capture images of an area outside the elongate body; and
at least one light emitter attached to the distal end of the elongate body and positioned to generate and direct light towards the area outside of the elongate body.
